(19) United States
(12) Patent Application Publication
(10) Pub. No.: US 2017/0143336 A1
(43) Pub. Date: May 25, 2017

(54) SURGICAL FASTENER APPLYING APPARATUS

(71) Applicant: Covidien LP, Mansfiekl, MA (US)

(72) Inventors: Sachin Shah, Milford, CT (US); Arvind Kuma Gupta, Uttar Pradesh (IN); Harshottam Singh Dhakad, Madhya Pradesh (IN); Manoj Kumar Agarwal, Ranchi Jharkhand (IN); Vinayan Vivekanandan, Kerala (IN)

(21) Appl. No.: 15/309,813

(22) PCT Filed: May 15, 2014
(86) PCT No.: PCT/US2014/038171

§ 371 Date: Nov. 9, 2016

(51) Int. Cl. A61B 17/072

(52) U.S. Cl. CPC A61B 17/07207; A61B 2017/07257; A61B 2090/0814

(57) ABSTRACT

A surgical fastener applying apparatus includes an anvil half-section (12′) including a distal anvil (12b′) and a proximal handle (12a′) defining a release aperture (185′) at its proximal end. A cartridge receiving half-section (14′) has a distal portion (24a′) dimensioned to receive a single use loading unit and a proximal portion (24b′) configured to support a firing assembly (20′). A clamping lever (16′) is secured to the cartridge receiving half-section and includes a handle portion (190′) supporting an engagement member (218′). A firing assembly is configured to be supported within the proximal portion of the cartridge receiving half-section. The clamping lever is movable from an undamped position to a clamped position to releasably secure the anvil of the anvil half-section in close approximation with the single use loading unit. In the clamped position, the engagement member of the clamping lever releasably engages the latch portion (210′) of the locking member (206′) and the release member (212′) extends at least partially into the release aperture.

### BACKGROUND

1. Technical Field

The present disclosure relates to a surgical fastener applying apparatus and, more particularly, to a surgical fastener applying apparatus having reusable and disposable components.

2. Discussion of Related Art

Surgical fastener applying apparatus, wherein tissue is first grasped or clamped between opposing jaw structures and then joined by means of surgical fasteners, are well known in the art. In some such apparatus, a knife is provided to transect the tissue which has been joined by the fasteners. The fasteners are typically in the form of surgical staples, although, other surgical fasteners may also be utilized, such as, for example, clips or two part polymeric surgical fasteners.

Surgical fastener applying apparatus typically include two elongated beam members which are used to capture or clamp tissue therebetween. Typically, one of the beam members carries a disposable cartridge assembly which houses a plurality of staples arranged in at least two lateral rows, while the other beam member includes an anvil which defines a surface for forming the staple legs as the staples are driven from the cartridge assembly. Where two part fasteners are used, the beam member which includes the anvil carries a mating part of the two part fastener, e.g. the receiver. Generally, the staple formation process is affected by the interaction between one or more longitudinally moving camming members and a series of individual staple pushers. As the camming members travel longitudinally through the cartridge carrying beam member, the individual pusher members are biased upwardly into a backspan of the staples supported within the cartridge assembly to sequentially eject the staples from the cartridge. A knife may be provided to travel with the camming members between the staple rows to transect the tissue between the rows of formed staples. An example of such an instrument is disclosed in U.S. Pat. No. 7,631,794, which is incorporated herein in its entirety by reference.

Because of the dangers associated with improper sterilization surgical fastener applying apparatus may be disposable after use. Although the cartridge assembly may be replaced to perform multiple fastener applying operations on a single patient, the surgical fastener applying apparatus may be disposable after a surgical procedure has been completed. This requirement of disposability may increase the costs associated with surgical procedures.

A need exists in the art for a fastener applying apparatus which includes reusable components, is not overly complex, and is configured to facilitate proper sterilization after use in a surgical procedure.

### SUMMARY

The present disclosure relates to a surgical fastener applying apparatus for applying surgical fasteners to body tissue. In embodiments, the surgical fastener applying apparatus includes an anvil half-section, a cartridge receiving half-section, a clamping lever, and a firing assembly. The anvil half-section includes a distal anvil portion and a proximal handle portion. A release aperture is defined at the proximal end of the proximal handle portion. The cartridge receiving half-section has a distal portion dimensioned to receive a single use loading unit and a proximal portion configured to support the firing assembly. The clamping lever is secured to the cartridge receiving half-section and has a proximal end and a distal end. The clamping lever includes a handle portion supporting an engagement member. The firing assembly is configured to be supported within the proximal portion of the cartridge receiving half-section and includes a stationary housing, a firing lever, a cam bar fixedly secured to the firing lever, and a pivotal locking member including a latch portion and a release member. The clamping lever is operably associated with the anvil half-section and the cartridge receiving half-section and is movable from an unclamped position to a clamped position to releasably secure the anvil portion of the anvil half-section in close approximation with the single use loading unit. In the clamped position, the engagement member of the clamping lever releasably engages the latch portion of the locking member and the release member extends at least partially into the release aperture.

In embodiments, the pivotal locking member includes a pair of pivot members which are pivotally supported in openings defined in a proximal end of the stationary housing of the firing assembly.

In embodiments, the release member and the latch portion of the pivotal locking member are disposed on opposite ends of the pivotal locking member. In such embodiments, the pivot members are disposed between the release member and the latch portion.

In embodiments, the proximal handle portion of the anvil half-section defines at least one cut-out adjacent the release aperture.

An embodiment of a surgical fastener applying apparatus provided in accordance with the present disclosure includes an anvil half-section, a cartridge receiving half-section, a clamping lever, and a firing assembly. The anvil half-section includes a distal anvil portion and a proximal handle portion. The distal anvil portion defines a longitudinally-extending knife slot and a transversely-extending slot. The knife slot and the transverse slot are disposed in substantially perpendicular orientation relative to one another and communicate with one another. The cartridge receiving half-section has a distal portion dimensioned to receive a single use loading unit and a proximal portion configured to support the firing assembly. The clamping lever is secured to the cartridge receiving half-section. The firing assembly is configured to be supported within the proximal portion of the cartridge receiving half-section and includes a stationary housing, a firing lever, a cam bar fixedly secured to the firing lever, and a knife actuating bar which is configured to engage a knife supported within the single use loading unit. The clamping lever is operably associated with the anvil half-section and the cartridge receiving half-section and is movable from an unclamped position to a clamped position to releasably secure the anvil portion of the anvil half-section in close approximation with the single use loading unit. In the clamped position, the firing lever is selectively advancable to advance the cam bar to eject staples from the single use loading unit and the knife actuating bar to translate the knife through the knife slot.

In embodiments, the proximal handle portion of the anvil half-section defines a plurality of longitudinally spaced cut-outs disposed along at least a portion of a length thereof.

In embodiments, the clamping lever includes a thumb abutment disposed towards the distal end thereof and further includes a pair of opposed cut-outs defined therethrough adjacent the thumb abutment.

Another surgical fastener applying apparatus provided in accordance with the present disclosure includes an anvil half-section, a cartridge receiving half-section, a clamping lever, and a firing assembly. The anvil half-section includes a distal anvil portion and a proximal handle portion. The cartridge receiving half-section has a distal portion dimensioned to receive a single use loading unit and a proximal portion configured to support the firing assembly. The clamping lever is secured to the cartridge receiving half-section. The clamping lever is operably associated with the anvil half-section and the cartridge receiving half-section and is movable from an unclamped position to a clamped position to releasably secure the anvil portion of the anvil half-section in close approximation with the single use loading unit. The firing assembly is configured to be supported within the proximal portion of the cartridge receiving half-section and includes a stationary housing, a firing lever, and a cam bar fixedly secured to the firing lever. The firing lever is being distally translatable from a proximal position to a distal position to advance the cam bar to eject staples from the single use loading unit and proximally translatable from the distal position to the proximal position to return the cam bar back to an initial position. The firing lever includes an engagement member having a first concave surface configured to facilitate finger engagement of the firing lever during distal translation of the firing lever and a second, opposed concave surface configured to facilitate finger engagement of the firing lever during proximal translation of the firing lever.

In embodiments, the firing assembly further includes a knife actuating bar which is configured to engage a knife supported within the single use loading unit to translate the knife through the single use loading unit upon translation of the firing lever.

In embodiments, the firing lever includes a pair of engagement members. A first engagement member extends from a first side of the firing assembly and a second engagement member extends from a second, opposite side of the firing assembly.

### BRIEF DESCRIPTION OF THE DRAWINGS

Various embodiments of the presently disclosed surgical fastener applying apparatus will now be described herein with reference to the accompanying figures wherein:

FIG. 1 is a side perspective view of an embodiment of the presently disclosed surgical fastener applying apparatus in the clamped position;

FIG. 2 is a side perspective view of the surgical fastener applying apparatus shown in FIG. 1 in the open position;

FIG. 2A is a side view of a portion of the cartridge receiving half-section and the clamping lever during assembly of the clamping lever and the cartridge receiving half-section;

FIG. 2B is a side perspective view of the fastener applying apparatus shown in FIG. 1 in the closed, unclamped position;

FIG. 3 is a side perspective view of the surgical fastener applying apparatus shown in FIG. 1 with parts separated;

FIG. 3A is a bottom, side cross-sectional view of the clamping lever of the surgical fastener applying apparatus shown in FIG. 1;

FIG. 4 is a side perspective view of the cartridge receiving half-section of the surgical fastener applying apparatus with the single use loading unit (“SULU”) and the firing assembly supported within the cartridge receiving half-section;

FIG. 5 is an enlarged view of the indicated area of detail shown in FIG. 4;

FIG. 6 is a perspective view from above of the cartridge receiving half-section of the surgical fastener applying apparatus with the SULU and the firing assembly supported therein;

FIG. 7 is an enlarged view of the indicated area of detail shown in FIG. 6;

FIG. 8 is a front end perspective view from above the firing assembly of the surgical fastener applying apparatus shown in FIG. 3;

FIG. 9 is an enlarged view of the indicated area of detail shown in FIG. 8;

FIG. 9A is a side perspective view of the channel member with the firing assembly releasably supported therein;

FIG. 9B is an enlarged view of the indicated area of detail shown in FIG. 9A;

FIG. 9C is a side perspective view of a central portion of the channel member;

FIG. 10 is a rear end perspective view from above of the firing assembly shown in FIG. 8;

FIG. 11 is an enlarged view of the indicated area of detail shown in FIG. 10;

FIG. 12 is a side perspective view of the firing assembly shown in FIG. 10 with parts separated;

FIG. 12A is a bottom, side perspective view of the cam bar of the firing assembly shown in FIG. 12;

FIG. 12B is a bottom, side perspective view of the firing lever of the firing assembly shown in FIG. 12;

FIG. 13 is a side perspective view of the SULU of the surgical fastener applying apparatus shown in FIG. 1;

FIG. 14 is an enlarged view of the indicated area of detail shown in FIG. 13;

FIG. 15 is a front perspective view of the SULU shown in FIG. 13;

FIG. 16 is an enlarged view of the indicated area of detail shown in FIG. 15;

FIG. 17 is a side perspective view with parts separated of the SULU shown in FIG. 15;

FIG. 18 is a side cross-sectional view of the surgical fastener applying apparatus shown in FIG. 1 in the open position;

FIG. 19 is an enlarged view of the indicated area of detail shown in FIG. 18;

FIG. 20 is an enlarged view of the indicated area of detail shown in FIG. 18;

FIG. 21 is a perspective view of the proximal end of the surgical fastener applying apparatus shown in FIG. 18 in the open position;

FIG. 22 is an enlarged view of the indicated area of detail shown in FIG. 18;

FIG. 23 is a perspective view of the proximal end of the clamping lever of the surgical fastener applying apparatus shown in FIG. 1;

FIG. 24 is a side perspective view of the surgical fastener applying apparatus shown in FIG. 1 in the clamped position;

FIG. 25 is a side cross-sectional view of the surgical fastener applying apparatus shown in FIG. 24 in the clamped position;

FIG. 26 is an enlarged view of the indicated area of detail shown in FIG. 25;

FIG. 27 is an enlarged view of the indicated area of detail shown in FIG. 25;

FIG. 28 is a cross-sectional view taken along section lines 28-28 of FIG. 26;

FIG. 29 is a top view of the surgical fastener applying apparatus shown in FIG. 1 as the firing assembly is moved through an actuating stroke to eject fasteners from the apparatus;

FIG. 30 is a side cross-sectional view of the surgical fastener applying apparatus shown in FIG. 29 with the firing assembly in the actuated position;

FIG. 31 is an enlarged view of the indicated area of detail shown in FIG. 30;

FIG. 32 is an enlarged view of the indicated are of detail shown in FIG. 30;

FIG. 33 is a side cross-sectional view of the surgical fastener applying apparatus shown in FIG. 1 after the apparatus has been fired and moved to the open position;

FIG. 34 is an enlarged view of the indicated area of detail shown in FIG. 33;

FIG. 35 is a side perspective view of an embodiment of the presently disclosed surgical fastener applying apparatus in the clamped position;

FIG. 36 is a side perspective view of the anvil half-section of the surgical fastener applying apparatus of FIG. 35;

FIG. 37 is a bottom perspective view of the distal end of the anvil half-section of the surgical fastener applying apparatus of FIG. 35;

FIG. 38 is a bottom perspective view of the proximal end of the anvil half-section of the surgical fastener applying apparatus of FIG. 35;

FIG. 39 is a side perspective view of the channel member of the surgical fastener applying apparatus of FIG. 35;

FIG. 40 is a side perspective view of the clamping lever of the surgical fastener applying apparatus of FIG. 35;

FIG. 41 is a transverse cross-sectional view of the clamping lever shown in FIG. 40;

FIG. 42 is a side perspective view of the firing assembly of the surgical fastener applying apparatus of FIG. 35 with parts separated; and

FIG. 43 is a transverse cross-sectional view of one of the finger engagement members of the firing assembly of the surgical fastener applying apparatus of FIG. 35.

### DETAILED DESCRIPTION OF EMBODIMENTS

Embodiments of the presently disclosed surgical fastener applying apparatus in accordance with the present disclosure will now be described in detail with reference to the drawings wherein like reference numerals identify similar or identical structural elements. As used herein, as is traditional, the term “proximal” refers to the end of the apparatus which is closer to the user and the term distal refers to the end of the apparatus which is further away from the user.

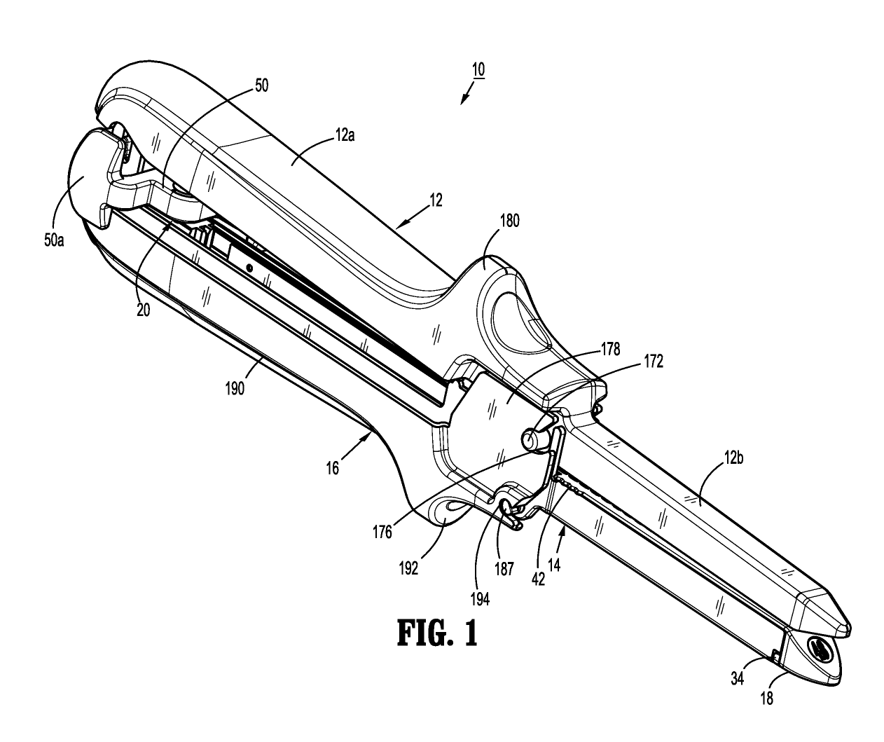

Referring to FIGS. 1-3, surgical stapler 10 includes an anvil half-section 12, a cartridge receiving half-section 14, a clamping lever 16, a single use loading unit 18 (hereinafter “SULU”) and a firing assembly 20. In an embodiment, anvil half-section 12, cartridge receiving half-section 14 and clamping lever 16 are constructed to be reusable components and, as such, are constructed from a biocompatible material suitable for sterilization and repeated use, e.g., stainless steel. In contrast, SULU 18 and firing assembly 20 are constructed to be disposable and, as such, may be constructed from any suitable biocompatible material, e.g., plastics, metals, combinations thereof, having the requisite strength characteristics.

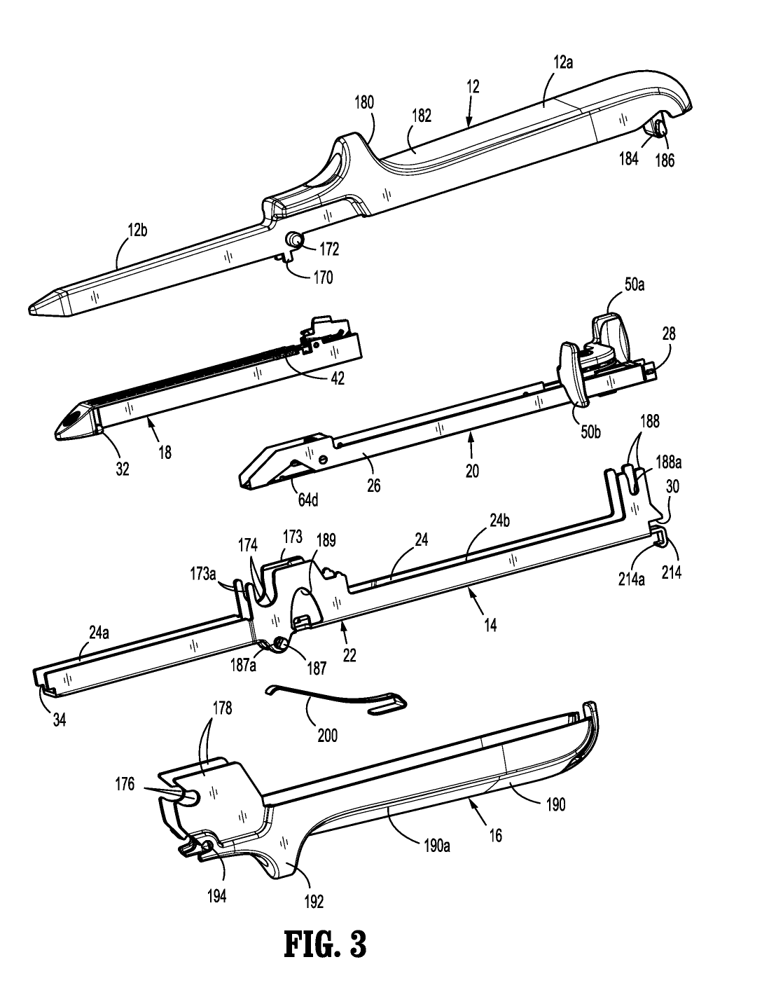

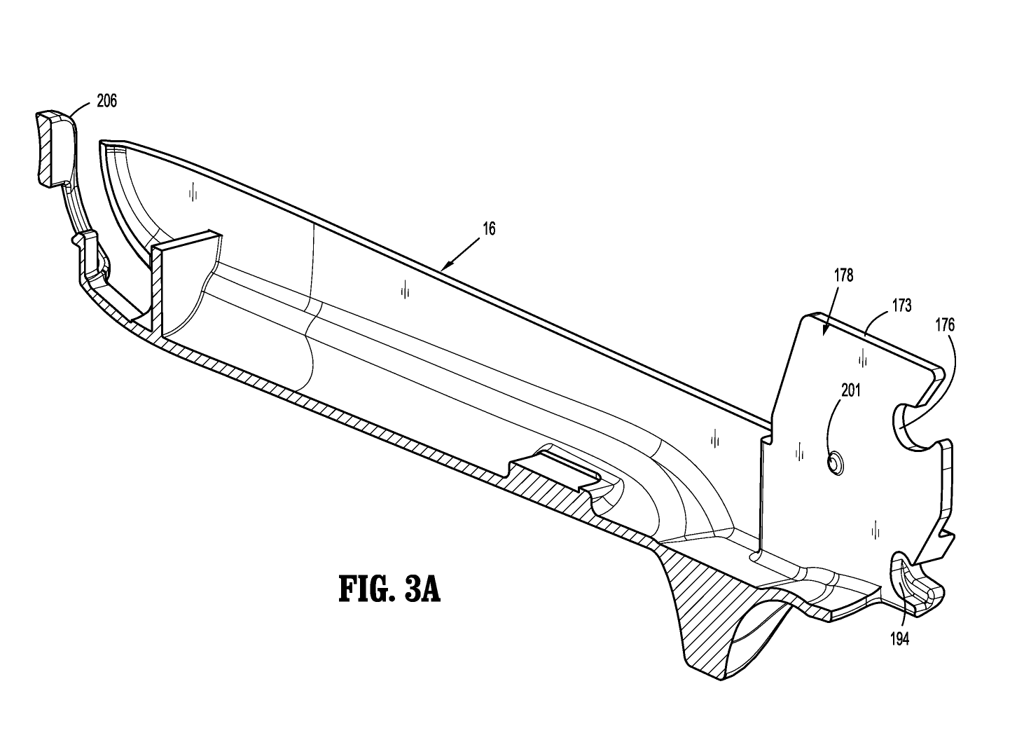

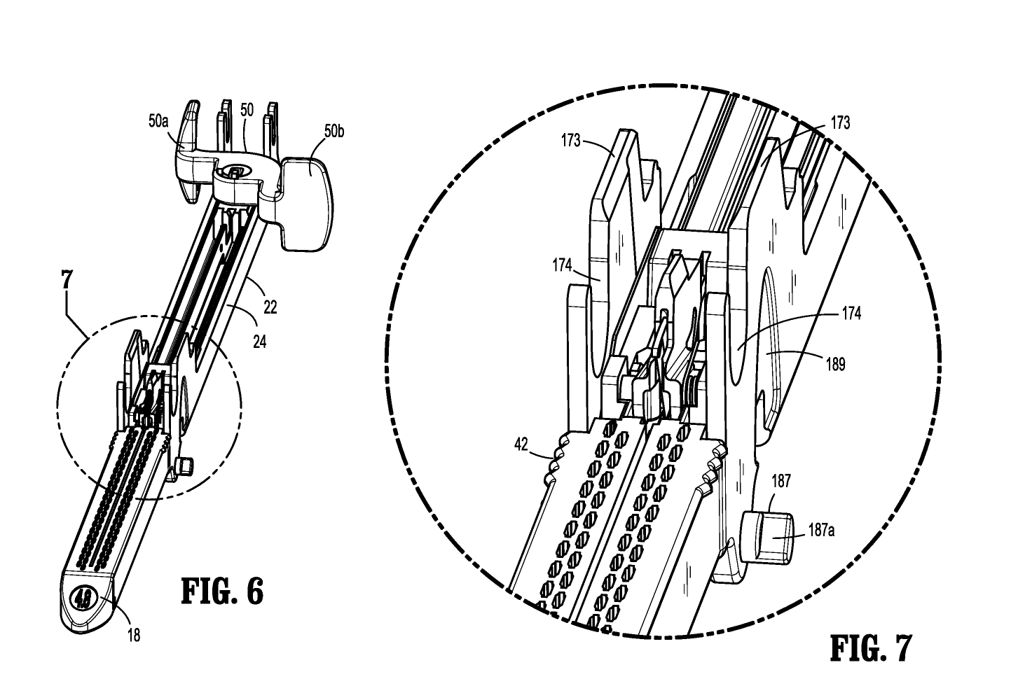

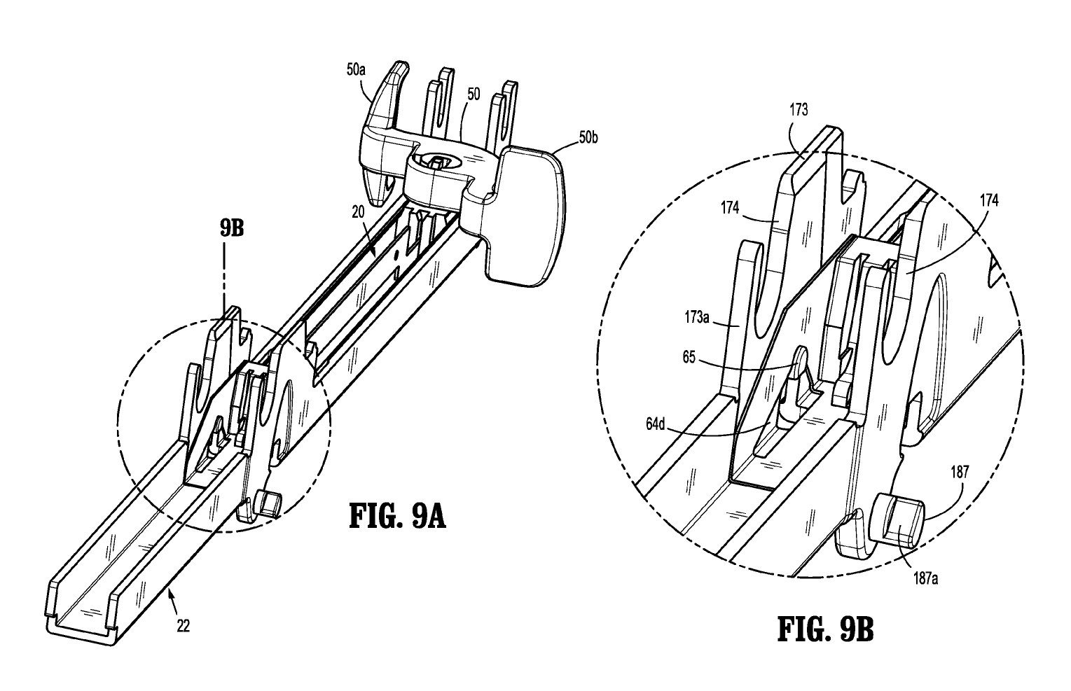

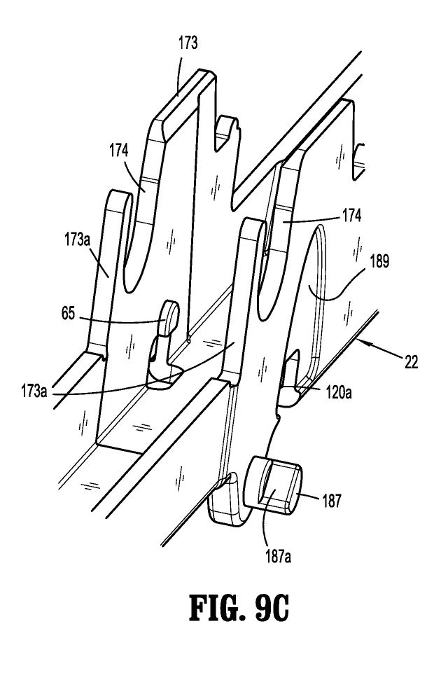

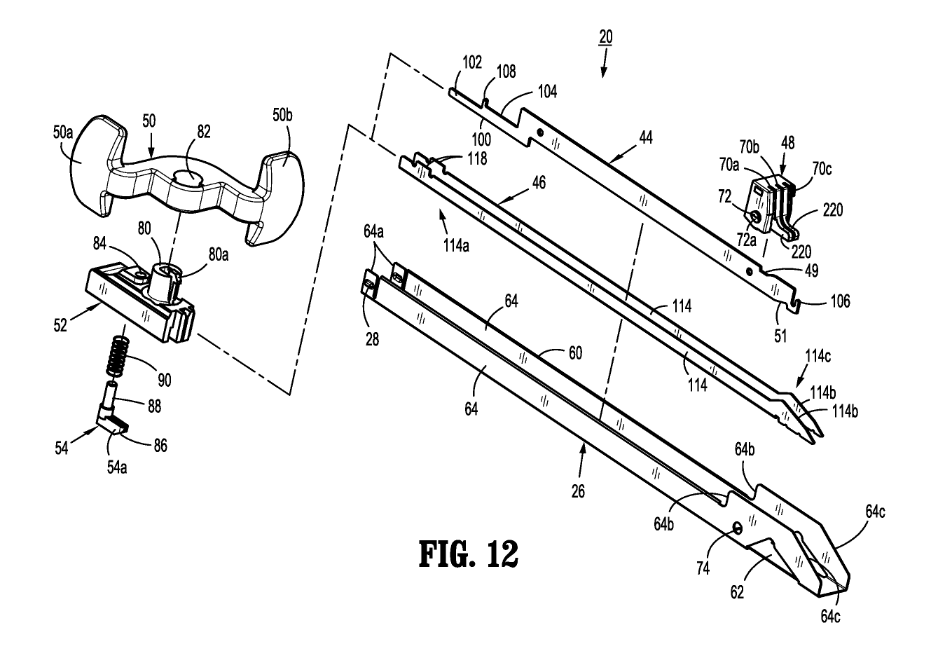

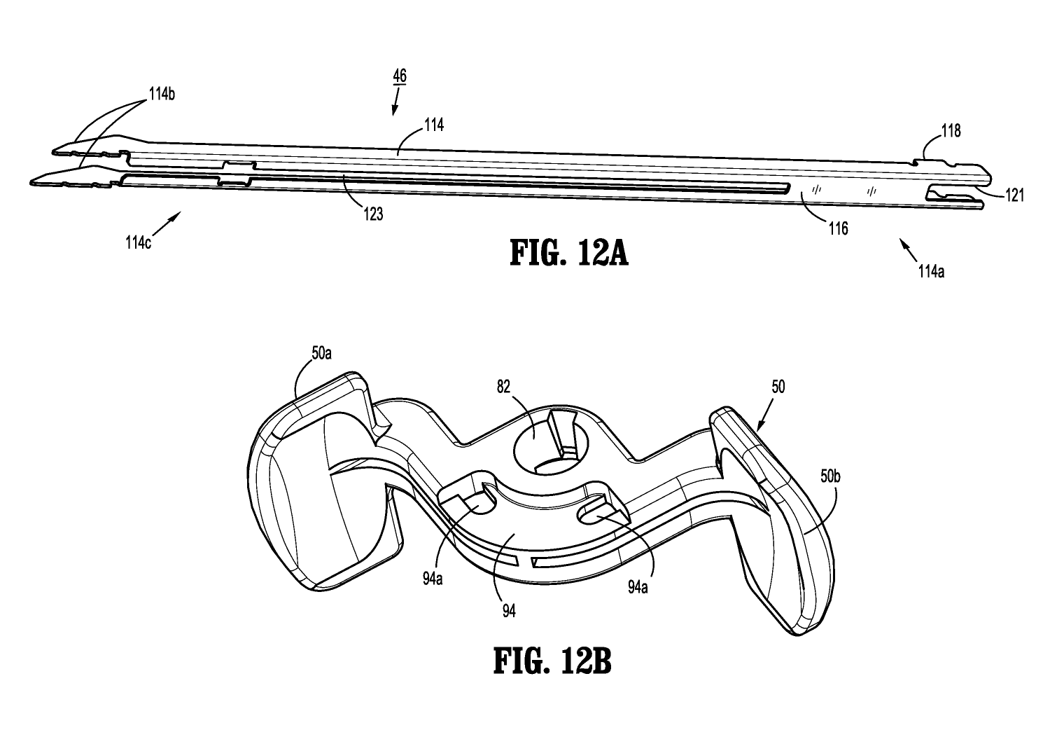

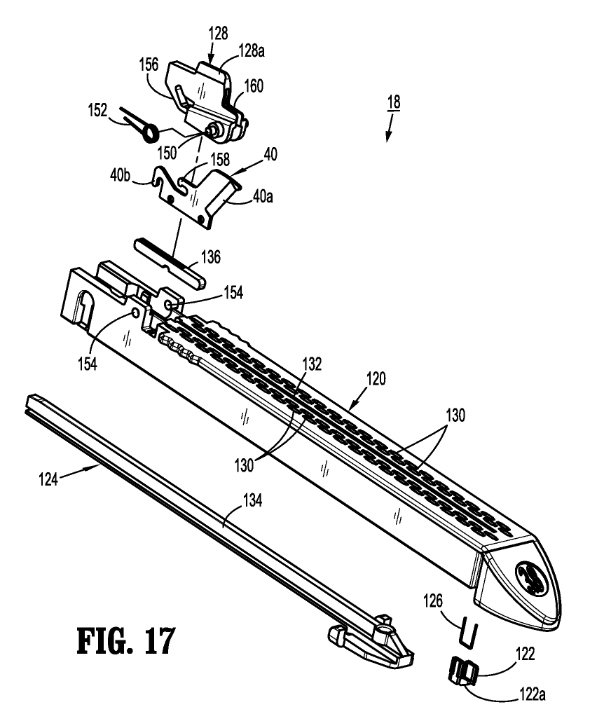

Referring to FIGS. 3-7, cartridge receiving half-section 14 defines an elongated channel member 22 which defines a substantially U-shaped channel 24 having a distal portion 24a dimensioned to releasably receive a SULU 18 and a proximal portion 24b dimensioned to releasably receive firing assembly 20. Firing assembly 20 includes a stationary housing 26 (see also FIG. 12) having a proximal end including protrusions 28 which extend into recesses 30 formed in a proximal portion of cartridge receiving half-section 14 to releasably secure the proximal end of firing assembly 20 within the proximal portion 24b of channel member 22. The distal end of firing assembly 20 defines a triangular cutout 64d which is positioned to receive a protrusion 65 formed on an inner wall of channel member 22 (see FIGS. 9A-9C) to releasably secure the distal end of firing assembly 20 within channel member 22. The structure of firing assembly 20 will be discussed in further detail below. Likewise, SULU 18 includes at least one protrusion such as a pair of distal protrusions 32 which are positioned in at least one aperture such as cutouts 34 formed at the distal end of channel member 22 to releasably secure SULU 18 within the distal portion 24a of channel member 22. During assembly, firing assembly 20 must be inserted into proximal portion 24b of channel member 22 before SULU 18 is inserted into distal portion 24a of channel member 22 as will be discussed below. To position SULU 18 in channel member 22, protrusions 32 on SULU 18 are positioned within cutouts 34 while SULU 18 is positioned above and at an angle to channel member 22. Thereafter, SULU 18 can be rotated downwardly into distal portion 24a of U-shaped channel 24. This allows for the drive components of firing assembly 20 to properly align with components of SULU 18 and also facilitates engagement of the firing assembly 20 with a knife 40 (FIG. 17) supported within SULU 18. A proximal end of SULU 18 includes an outwardly extending serrated surface 42 (FIG. 7) to facilitate gripping of the proximal end of SULU 18 to allow for removal and/or replacement of SULU 18 from channel member 22. Prior to movement of stapler 10 to the clamped position, as will be discussed below, serrated gripping surface 42 will not fully seat within distal portion 24a of channel member 22.

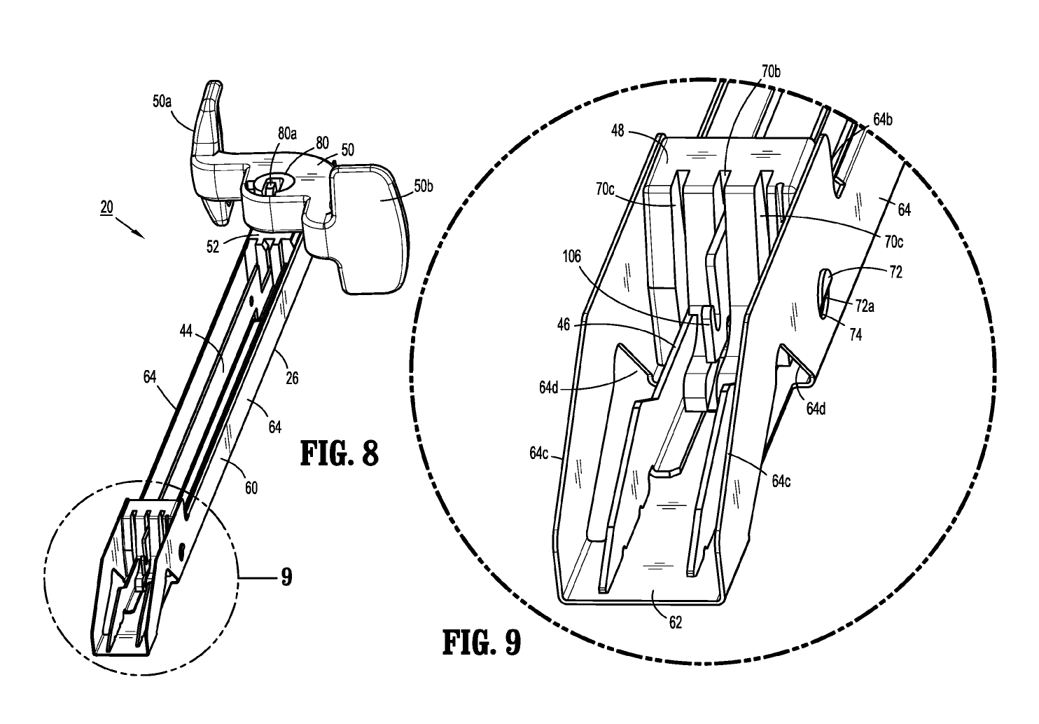

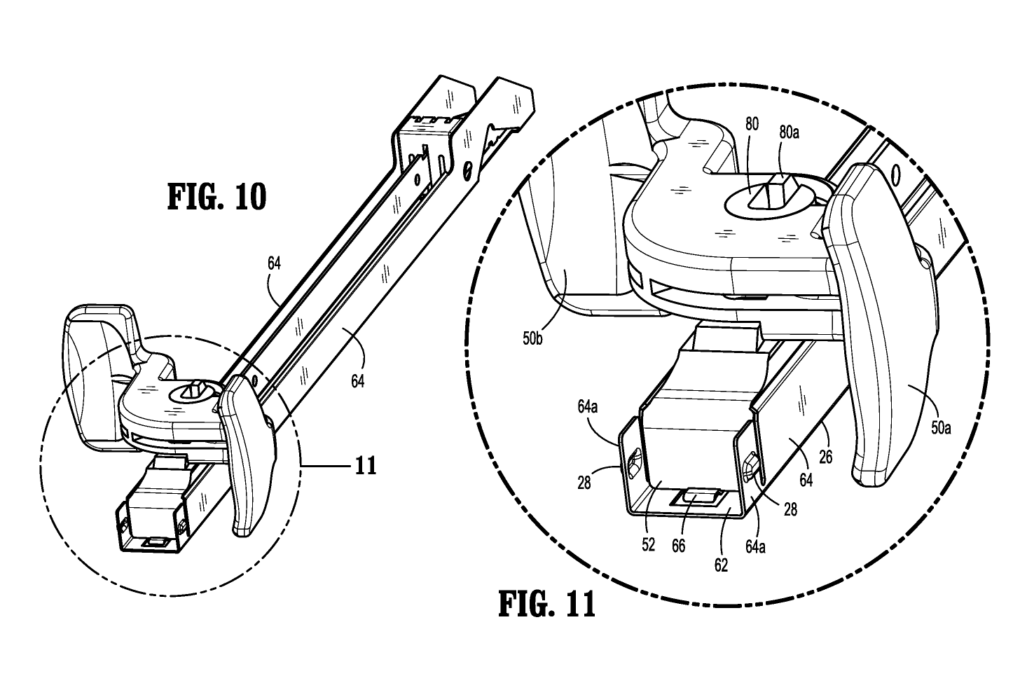

Referring to FIGS. 8-12, firing assembly 20 includes stationary housing 26, a knife actuating bar 44, a cam bar 46, a guide block 48, a firing lever 50, a slide block 52 and a pedal 54 (FIG. 12). In an embodiment, stationary housing 26 includes a U-shaped frame 60 including a bottom wall 62 and a pair of sidewalls 64. Each sidewall 64 is slotted at its proximal end to define a cantilevered section 64a (FIG. 11) which supports a respective protrusion 28. Cantilevered sections 64a allow protrusions 28 to flex inwardly into recesses 30 of channel member 22 (FIG. 21) to releasably secure firing assembly 20 within proximal portion 24b (FIG. 3) of channel member 22 as discussed above. The distal end of each sidewall 64 defines a proximal step 64b, a distal angled portion 64c (FIG. 9) and the triangular cutout 64d. As discussed above, triangular cutout 64d is positioned to receive a protrusion 65 (FIG. 9B) formed on an inner wall of channel member 22 to retain the distal end of firing assembly 20 within channel member 22. Bottom wall 62 includes a spring arm 66 (FIG. 11) which engages a proximal end of slide block 52 to prevent proximal movement of slide block 52 beyond a predetermined point within stationary housing 26.

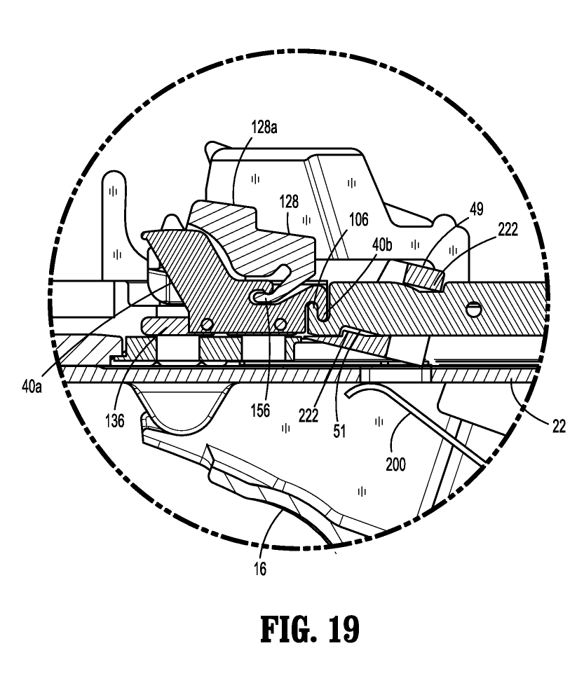

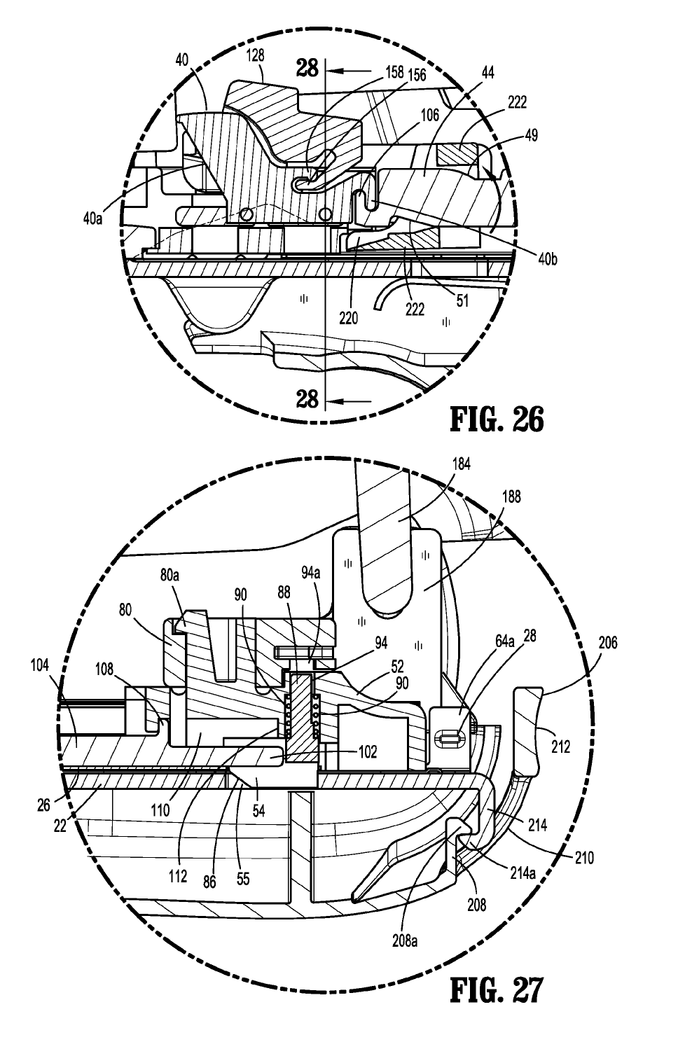

Guide block 48 includes a body defining three longitudinal slots 70a-c and a pair of outwardly extending protrusions 72. In an embodiment, each protrusion 72 is substantially cylindrical and includes a tapered portion 72a (FIG. 9). Alternately, other protrusion configurations are envisioned. Protrusions 72 are dimensioned to be received in openings 74 (FIG. 12) formed in sidewalls 64 of stationary housing 26 to axially fix guide block 48 within the distal end of stationary housing 26. Protrusions 72 allow for a degree of pivotal movement of guide block 48 within U-shaped frame 60. As will be discussed in further detail below, guide block 48 is pivotal from a first position (FIG. 19) in locking engagement with notches 49 and 51 of knife actuating bar 44 to a second position (FIG. 26) disengaged from notches 49 and 51 of knife actuating bar 44 in response to movement of stapler 10 to the clamped position. Each of slots 70a and 70c is dimensioned to slidably receive a respective sidewall 114 of cam bar 46. Similarly, slot 70b is dimensioned to slidably receive knife actuating bar 44. It is envisioned that guide block 48 may include any number of longitudinal slots.

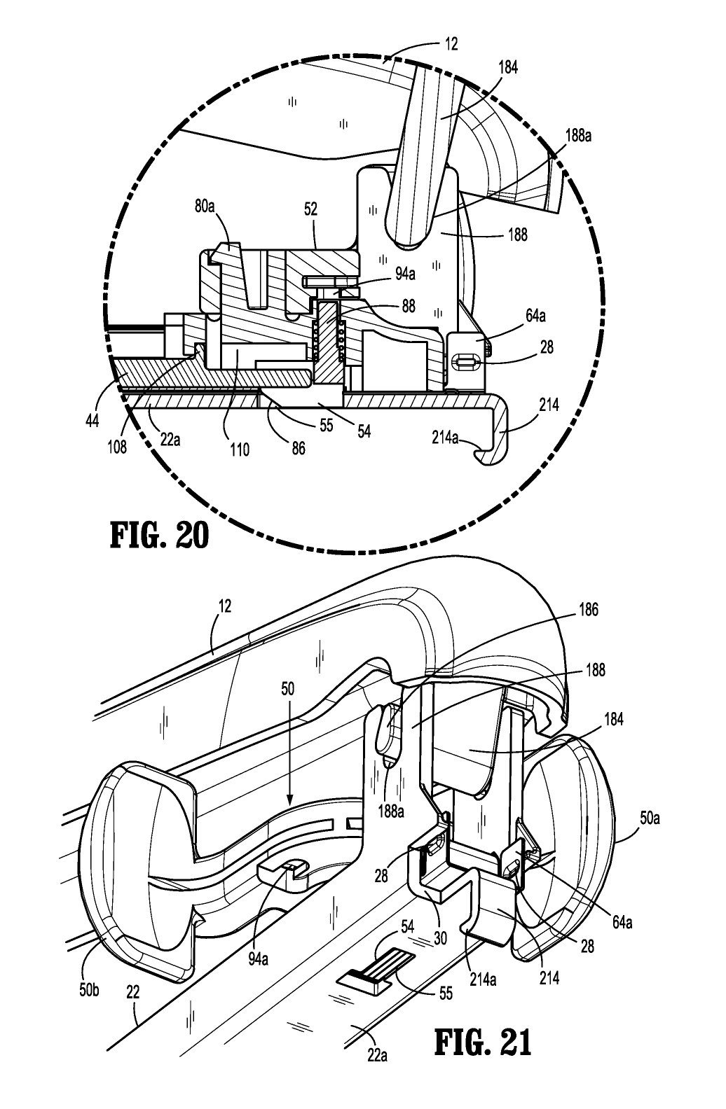

Slide block 52 includes a hub 80 which includes a resilient finger 80a configured to be snap-fit into a pivot hole 82 formed in firing lever 50. It is also envisioned that hub 80 may be retained within pivot hole 82 by any number of known ways, such as frictionally fitted of via an interlocking feature. Firing lever 50 is pivotal about hub 80 when the slide block 52 is in a retracted position to facilitate actuation of the firing assembly 20 from either side of stapler 10. Pedal 54 is reciprocally received within a hole 84 formed in slide block 52. Pedal 54 may include a split body portion 54a which is configured to straddle a proximal end 102 of knife actuating bar 44. In an embodiment, split body portion 54a includes an angled distal surface 86. A pin 88 extends upwardly from pedal 54 through hole 84 in slide block 52. A biasing member 90 is positioned between split body portion 54a and slide block 52, about pin 88 to urge pedal 54 downwardly away from slide block 52. In the retracted position of slide block 52, pedal 54 is received in a cutout 55 formed in a bottom wall 22a of channel member 22 (FIG. 20).

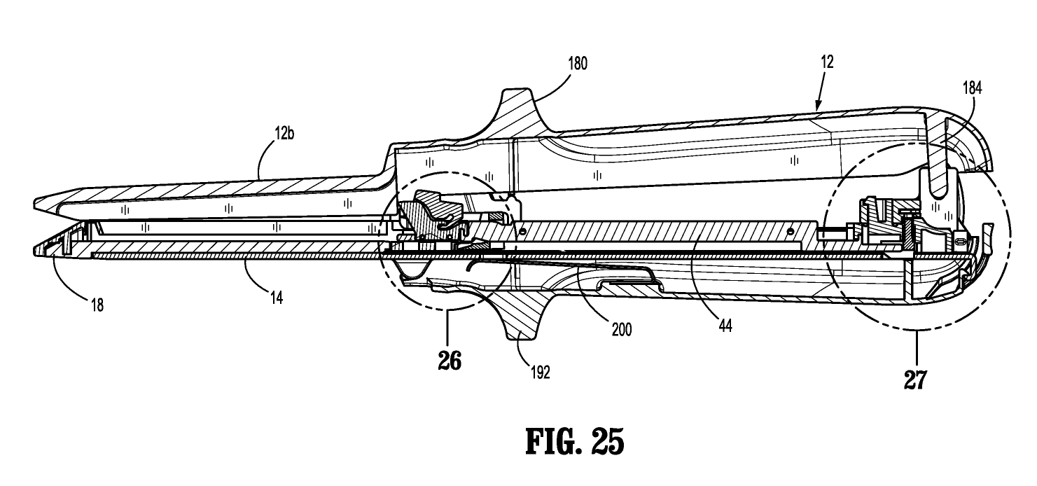

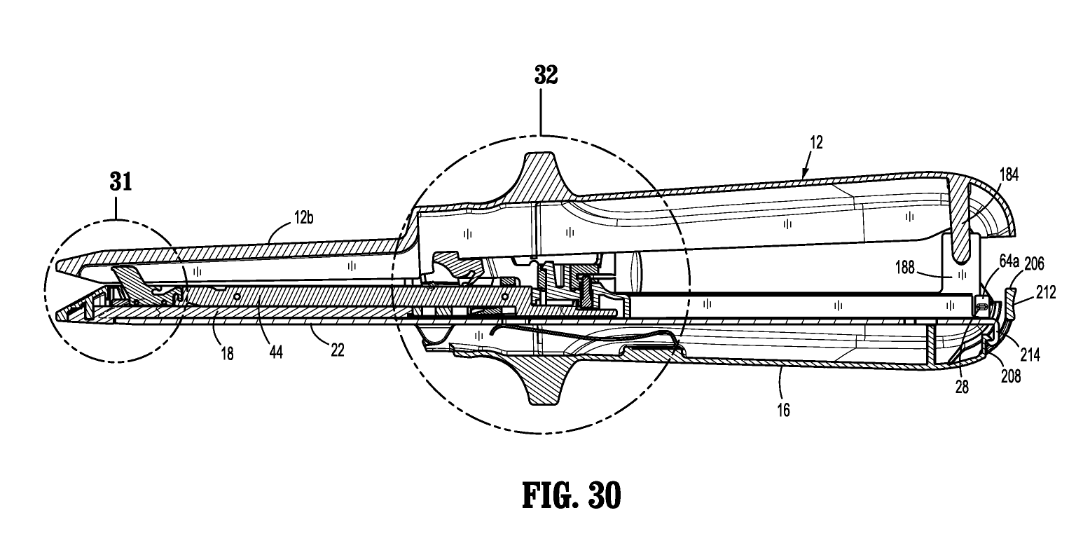

Firing lever 50 includes a plurality of engagement members such as first and second finger engagement members 50a and 50b, either one of which can be selectively engaged to move the firing lever 50 through a firing stroke from either side of stapler 10. An arcuate recess 94 (FIG. 12B) is formed in a bottom surface of firing lever 50 which slidably receives pin 88 of pedal 54 to define the range of rotation through which firing lever 50 can pivot about hub 80 of slide block 52. As used herein, a firing stroke is defined as movement of firing lever 50 from a fully retracted position (FIG. 25) to a fully advanced position (FIG. 30). A stop recess 94a is formed at each end of arcuate recess 94. Stop recesses 94a are configured and dimensioned to receive the end of pin 88 of pedal 54 to prevent pivotal movement of firing lever 50 about hub 80 during a firing stroke of surgical stapler 10. More specifically, when the firing assembly 20 is actuated to advance slide block 52 distally within stationary housing 26, angled distal surface 86 of pedal 54 engages channel member 22 and is cammed out of cutout 55 (FIG. 27) to urge pin 88 upwardly into a stop recess 94a to prevent pivotal movement of firing lever 50 during movement of firing lever 50 through a firing stroke. As is evident, pin 88 must be positioned beneath a stop recess 94a to allow pedal 54 to lift upwardly from cutout 55 to allow firing lever 50 to be moved through the firing stroke. Thus, firing lever 50 must be pivoted to one side or the other of firing assembly 20 before the firing lever 50 can be moved through a firing stroke.

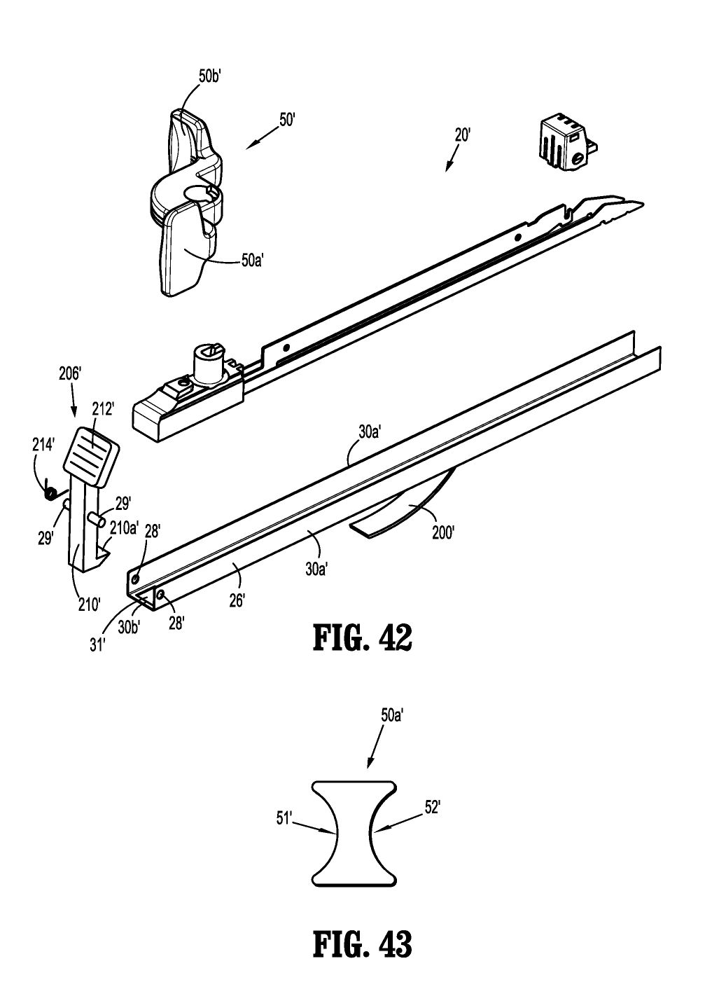

With reference to FIGS. 42 and 43, in an embodiment, first and second finger engagement members 50a′ and 50b′ of firing lever 50′ of firing assembly 20′ may be ergonomically configured to facilitate the distal advancement and proximal return of firing lever 50′ during movement of the firing lever 50′ through a firing stroke and return stroke. As best shown in FIG. 43, engagement member 50a′ may define opposed concave surfaces 51′, 52′, thus defining an overall hourglass-like configuration (in cross-section). Concave surface 51′ receives the operator's finger during distal advancement of firing lever 50′ and, as a result of its concave shape, inhibits the operator's finger from slipping off of engagement member 50a′ during advancement of firing lever 50′. Likewise, concave surface 52′ receives the operator's finger during proximal return of firing lever 50′ and inhibits the operator's finger from slipping off of engagement member 50a′ during return of firing lever 50′. Engagement member 50b′ may be configured similarly to engagement member 50a′. It is envisioned that first and second finger engagement members may be any number of shapes, such as flat, arcuate or ring-shaped.

Referring again to FIGS. 8-12, knife actuating bar 44 includes a proximal end having a stepped portion 100 which includes a proximal first step 102 having a first height and a second step 104 having a second height which is greater than the first height. A distal end of actuating bar 44 includes an upturned hook portion 106 and upper and lower notches 49 and 51. A finger 108 projects upwardly from knife actuating bar 44 between first and second steps 102 and 104. As shown in FIG. 27, finger 108 is slidably received within a recess 110 formed in an underside of slide block 52. When slide block 52 is advanced distally within stationary housing 26, finger 108 moves within recess 110 such that slide block 52 moves in relation to knife actuating bar 44 until finger 108 engages a wall 112 (FIG. 32) defining a proximal end of recess 110. When finger 108 engages wall 112, further distal movement of slide block 52 will also effect distal movement of knife actuating bar 44. As will be evident below, this arrangement allows for staples to be ejected from SULU 18 prior to transecting tissue.

Referring to FIGS. 12 and 12A, cam bar 46 includes a pair of sidewalls 114 and a base wall 116 (FIG. 12A). The proximal end 114a of each sidewall 114 includes a raised wall portion 118. Each raised wall portion 118 is configured to be fixedly received in a slot (not shown) formed in an underside of slide block 52 to fixedly secure the proximal end of cam bar 46 to slide block 52. Alternately, slide block 52 may be molded about the proximal end of knife actuating bar 44. The distal end of each sidewall 114 includes an angled camming surface 114b. Base wall 116 defines a distally extending elongated slot 123 (FIG. 12A) which extends from the distal end of cam bar 46 along a substantial length of the cam bar 46 and a proximally extending longitudinal slot 121. Slot 121 is positioned to facilitate the passage of pedal 54 through cutout 55 of channel member 22 when slide block 52 is in the retracted position. See FIG. 27.

Sidewalls 114 of cam bar 46 are slidably positioned in slots 70a and 70c of guide block 48 and knife actuating bar 44 is slidably positioned in longitudinal slot 70b of guide block 48. When firing assembly 20 is supported in channel member 22 and firing lever 50 is pivoted to one side of stationary housing 26 and pushed distally, slide block 52 is moved distally within stationary housing 26. As slide block 52 begins to move distally, tapered surface 86 of pedal 54 engages a distal edge of channel member 22 defining cutout 55 to urge pedal 54 upwardly out of cutout 55, through slot 121 of cam bar 46, and onto an inner surface of stationary housing 26 of firing assembly 20 (FIG. 27). As this occurs, pin 88 of pedal 54 moves into a stop recess 94a to prevent further pivotal movement of firing lever 50. As firing lever 50 is moved distally, finger 108 moves within recess 110 such that knife actuating bar 44 remains stationary as cam bar 46 is advanced distally. When finger 108 engages proximal wall 112 defining recess 110, knife actuating bar 44 is moved distally with slide block 52 and cam bar 46. As will be discussed below, when cam bar 46 and knife actuating bar 44 are moved distally within stationary housing 26 of firing assembly 20 and channel member 22, angled camming surfaces 114b of cam bar 46 are moved through SULU 18 to eject fasteners from SULU 18. Simultaneously, although with a preset delay equal to the length of recess 112 (FIG. 32), knife actuating bar 44 drives a knife blade 40 through SULU 18 to transect tissue.

U.S. Pat. No. 7,631,794 (“the '794 patent”) discloses an exemplary surgical fastener applying apparatus which includes a firing assembly similar to that described above. The '794 patent is incorporated herein by reference in its entirety.

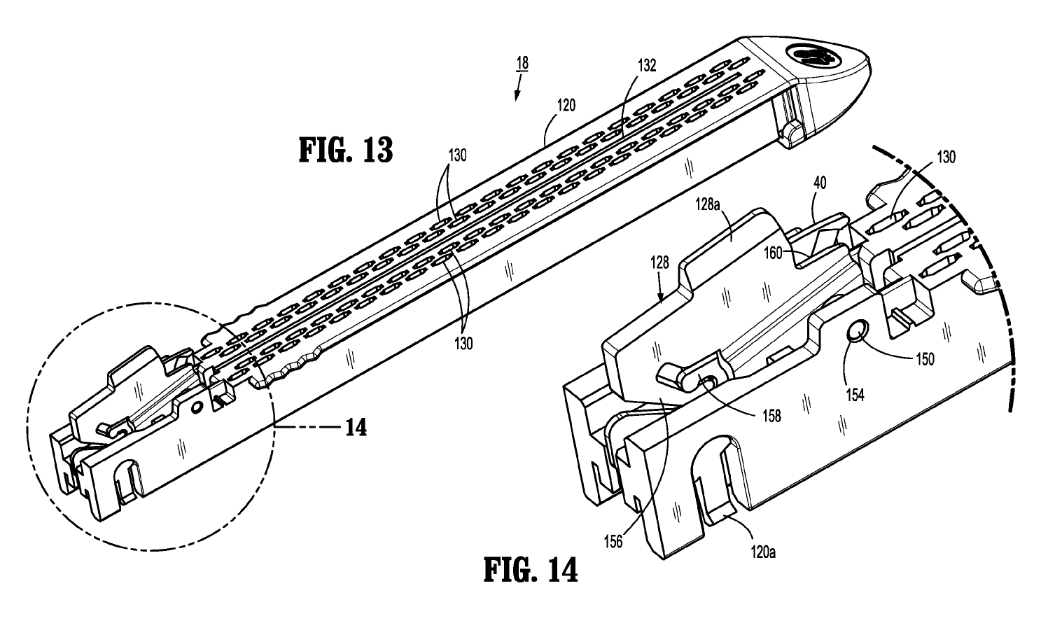

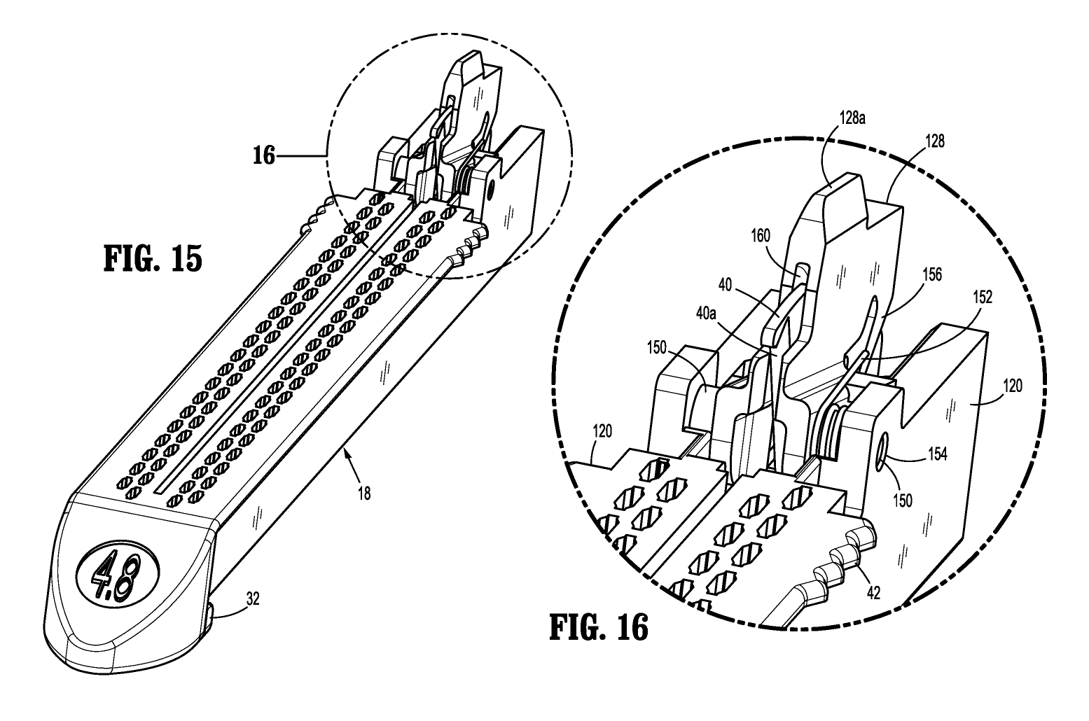

FIGS. 13-17 illustrate SULU 18. Referring to FIG. 17, SULU 18 includes a body 120, a plurality of staple pushers 122 (only one is shown), a bottom cover 124, a knife 40 having an angled sharpened leading edge or blade 40a, a plurality of staples 126 (only one is shown), and a pivotally mounted safety lockout 128. A proximal end of body 120 includes a flexible finger 120a which projects slightly beyond the outer wall defining body 120. Finger 120a frictionally engages an inner wall of channel member 22 to retain the proximal end of SULU 18 within channel member 22 when SULU 18 is releasably positioned within channel member 22. As is known in the art, body 120 has a plurality of rows of staple retaining slots 130, e.g., four, six, etc. and a linear slotted knife track 132 centrally disposed in body 120. Surgical stapler 10 can be dimensioned to receive or accommodate SULU's of different staple line lengths including, e.g., 60 mm, 80 mm and 100 mm. Knife 40 includes a downturned hook portion 40b which is positioned to engage upturned hook portion 106 of knife actuating bar 44 when SULU 18 is positioned within channel member 22.

In the illustrated embodiment, body 120 includes two staggered rows of slots 130 formed on either side of linear slotted knife track 132. The staggered rows of slots 130 extend beyond the distal end of knife track 132 to facilitate staple formation beyond the distal end of the stroke of the knife blade 40. Other configurations of slots 130 are contemplated, such as unstaggered or having multiple rows on at least one side of linear slotted knife track 132.

Staple pushers 122 may be configured to extend into one or more slots 130. In an embodiment, a single pusher is associated with each slot 130. Alternately, as illustrated in FIG. 17, each pusher 122 can be configured to extend into two adjacent slots 130 and is positioned beneath respective staples 126 which are retained in slots 130. As is known in the art, each pusher 122 includes a lower cam surface 122a which is positioned to engage one of cam surfaces 114b on the distal end of cam bar 46 such that movement of cam bar 46 through SULU 18 sequentially lifts each respective pusher 122 within its respective slot or slots 130 to eject staples from slots 130.

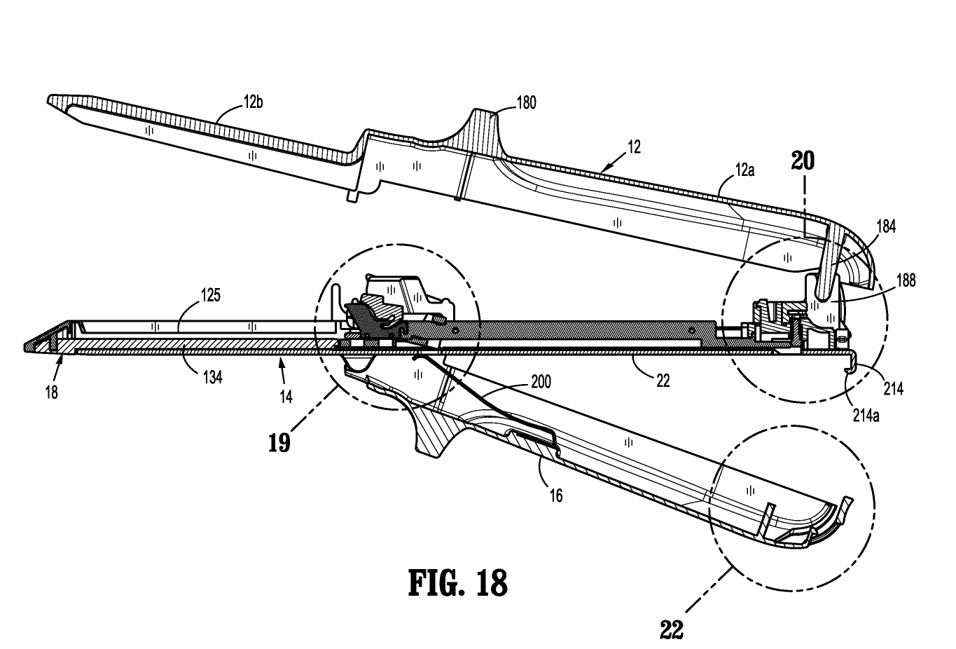

Bottom cover 124 partially encloses a channel 125 (FIG. 18) formed within the cartridge body 120. A longitudinal ridge 134 is formed on an upper surface of bottom cover 124 and provides a bearing surface for a knife supporting member 136 which is secured to a bottom edge of knife 40. Knife 40 may be secured to supporting member 136 via screws, pins, welding, adhesive or other known fastening techniques. During a firing stroke, knife 40 is guided along knife track 132 as the firing lever 50 is advanced through channel member 22. A pair of slots 138 are defined between the sides of ridge 134 and an outer wall of cartridge body 120. Longitudinal ridge 134 is positioned within body 120 and dimensioned to be slidably received in elongated slot 120 (FIG. 12a) of cam bar 46 such that cam bar 46 is slidably movable through cartridge body 120 about longitudinal ridge 134 to eject staples 126 from SULU 18.

Safety lockout 128 may be pivotally disposed on an upper proximal end of body 120 and is pivotal about a pivot member 150 from a locked orientation (FIG. 26) to unlocked orientation (FIG. 34). Pivot member 150 is received in openings 154 in body 120. A biasing member, e.g., spring 152, is positioned between knife supporting member 136 and safety lockout 128 to urge safety lockout 128 towards the unlocked orientation. Safety lockout 128 includes a proximal hook 156 which is positioned to receive an engagement member 158 formed on the knife 40 to retain the safety lockout 128 in the locked orientation when the knife 40 is in the retracted position (FIG. 19). When the knife 40 is moved towards the advanced position during a firing stroke, engagement member 158 is moved away from proximal hook 156 to allow safety lockout 128 to pivot towards the unlocked position in response to the urging of spring 152. It is noted that safety lockout 128 is prevented from pivoting to the unlocked position when the anvil half-section 12 and cartridge receiving half-section 14 are in the clamped position because the top surface 128a of safety lockout 128 engages an inner surface of anvil half-section 12 to prevent pivoting of safety lockout 128. Safety lockout 128 defines a slot 160 dimensioned to slidably receive the knife 40. In the retracted position of the knife 40, the leading edge 40a of knife 40 is confined within slot 160 safety lockout 128 to prevent accidental engagement and injury to medical personnel with leading edge 40a of knife 40.

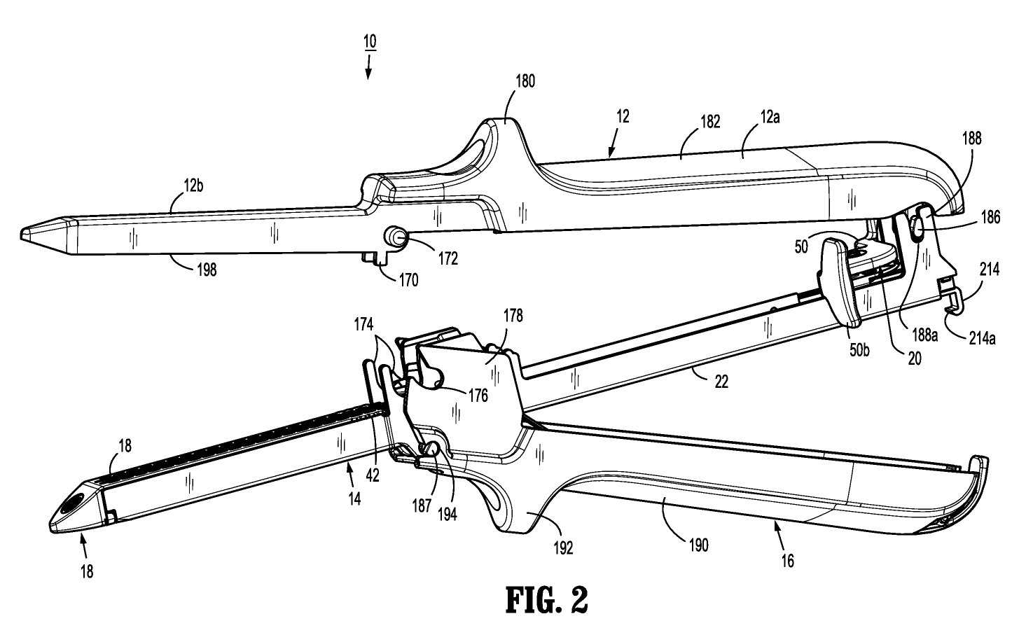

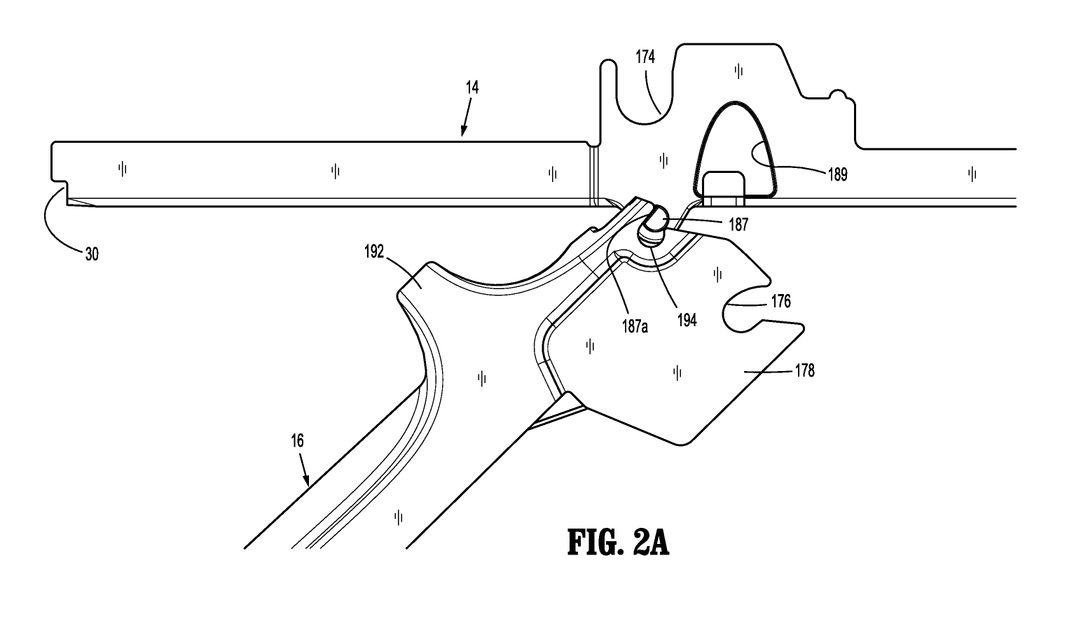

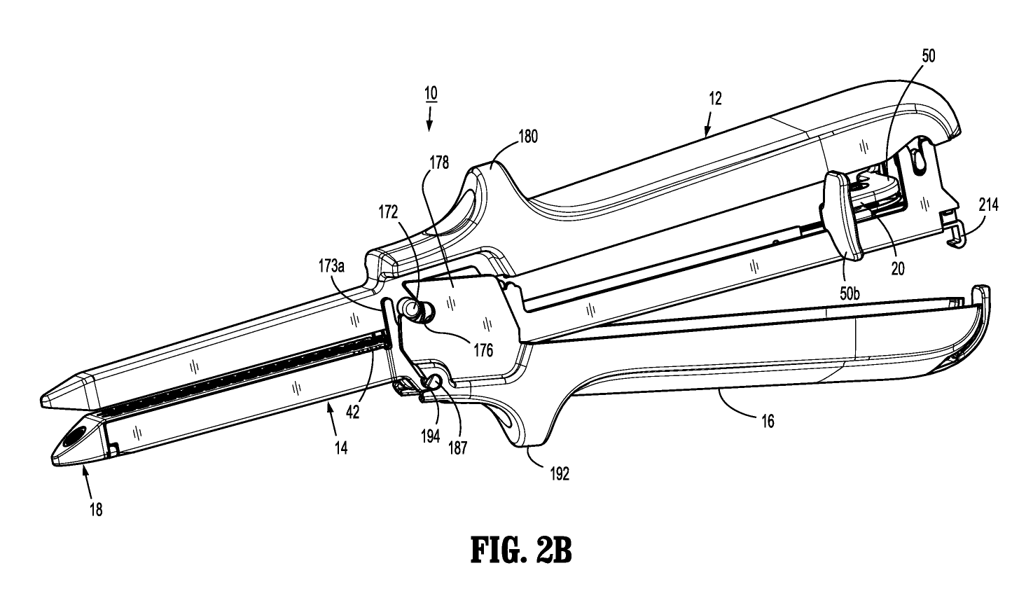

Referring again to FIGS. 2-3, anvil half-section 12 includes a proximal handle portion 12a and a distal anvil portion 12b. Anvil portion 12b includes a staple deforming portion 198 which, as known in the art, includes a plurality of staple deforming recesses and faces a top surface of SULU 18 when SULU 18 is positioned in the channel member 22. As is also known in the art, the staple deforming portion 198 includes a central longitudinal slot 15a′ (FIG. 37) for receiving the knife 40 (FIG. 17) as the knife 40 is moved through the SULU 18. The staple deforming portion 198 can be formed integrally with anvil half-section 12, or in the alternative, secured to anvil half-section 12 by a fastening process such as welding. A pair of locating fingers 170 are positioned adjacent the proximal end of the staple deforming portion 198 of anvil portion 12b and function to properly align SULU 18 with staple deforming portion 198.

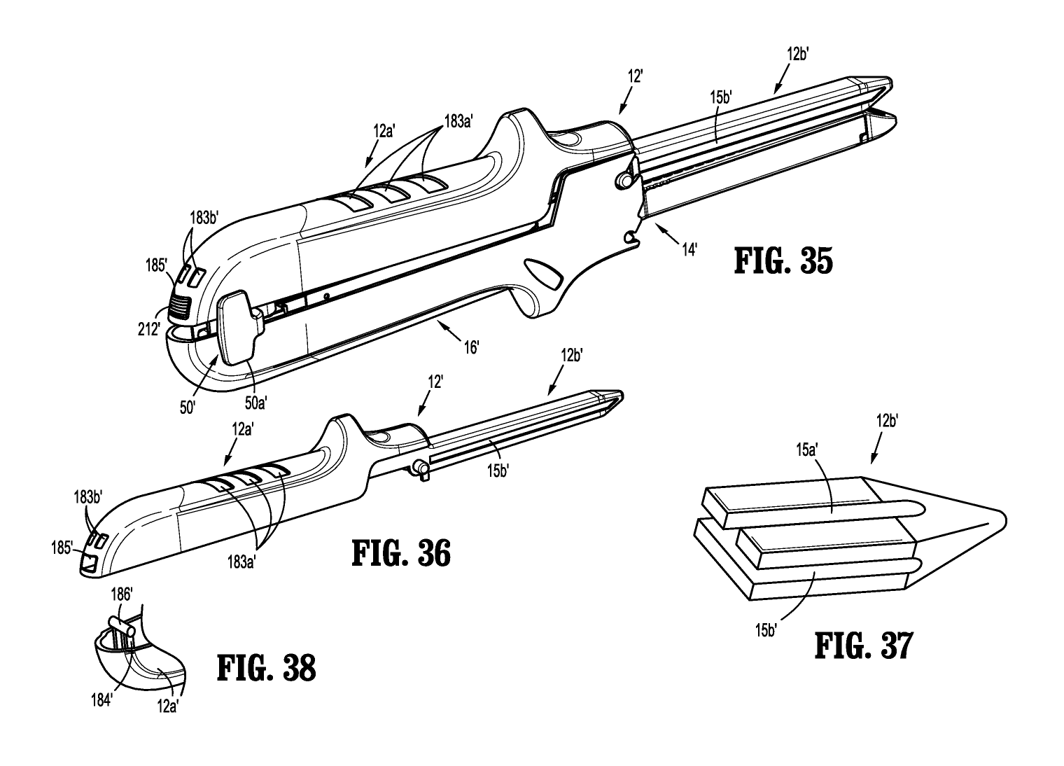

With reference to FIGS. 35-37, in some embodiments, anvil portion 12b′ of anvil half-section 12′ may include an elongated transverse slot 15b′ defined through anvil portion 12b′ and extending substantially along the entire length of anvil portion 12b′. Elongated transverse slot 15b′ extends transversely through anvil portion 12b′ such that elongated transverse slot 15b′ is open on either side of anvil portion 12b′, thus providing access to the interior of anvil portion 12b′ from either side of anvil portion 12b′. Elongated transverse slot 15b′ communicates with central longitudinal slot 15a′ of anvil portion 12b′ which, as mentioned above, is configured for receiving the knife 40 (FIG. 17) as the knife 40 (FIG. 17) is moved through the SULU 18 (FIGS. 2-3). Elongated transverse slot 15b′ and central longitudinal slot 15a′ are disposed in substantially perpendicular orientation relative to one another, thus defining a generally T-shaped opening (in cross-section) extending longitudinally though anvil portion 12b′. Providing elongated transverse slot 15b′ extending through anvil portion 12b′ and in communication with central longitudinal slot 15a′ facilitates cleaning and sterilization of anvil portion 12b′ and reduces the overall amount of material required to form anvil half-section 12′ and the weight of anvil half-section 12′, while maintaining the requisite strength and functionality thereof.

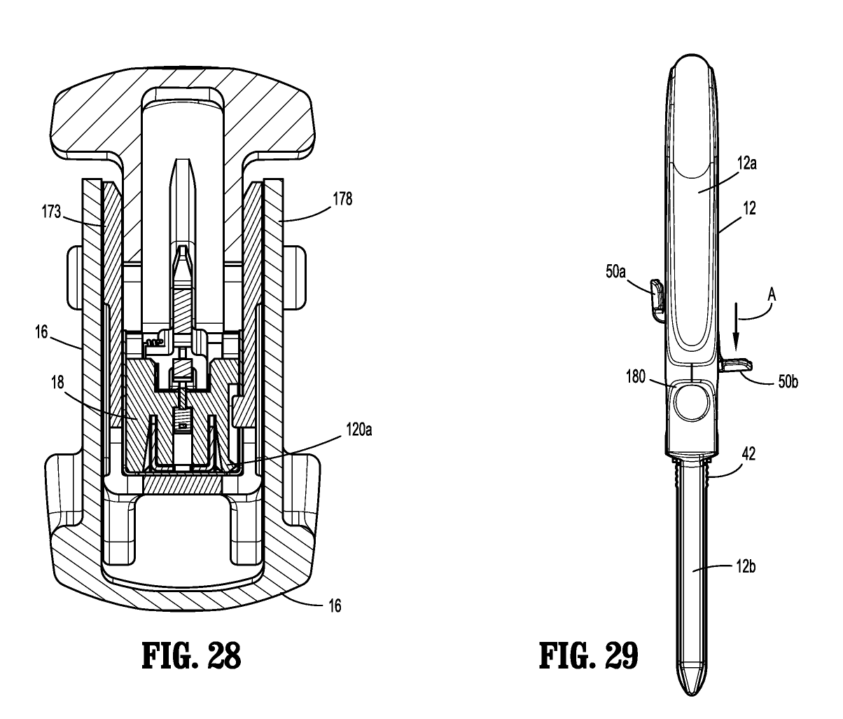

Referring again to FIGS. 2-3, a central portion of anvil half-section 12 includes a pair of cylindrical lateral support members 172. During assembly of anvil half-section 12 and cartridge receiving half-section 14, lateral support members 172 are supported in U-shaped recesses 174 formed on a central portion 173 of cartridge receiving half-section 14 (FIG. 28). Lateral support members 172 are also positioned to be received in cutouts 176 formed on spaced flange portions 178 of clamping lever 16 when the clamping lever 16 is moved to the clamped position (FIG. 2B). Proximal handle portion 12a is ergonomically formed and includes a thumb-engaging abutment 180 and a gripping portion 182. A proximal end of handle portion 12a includes a downwardly extending finger 184 which includes a pair of opposed teardrop shaped protrusions 186 which will be discussed in further detail below. Alternately, protrusions 186 may assume a variety of configurations.

As shown in FIGS. 35 and 36, in some embodiments, proximal handle portion 12a′ of anvil half-section 12′ may include a plurality of longitudinally-spaced cut-outs 183a′ defined through gripping portion 182′ of proximal handle portion 12a′ and extending along at least a portion of the length of proximal handle portion 12a′. Cut-outs 183a′ facilitate cleaning and sterilization of proximal handle portion 12a′, reduce the overall amount of material required to form anvil half-section 12′, and reduce the weight of anvil half-section 12′, while maintaining the requisite strength and functionality thereof.

Alternatively or additionally, proximal handle portion 12a′ may include a pair of laterally-spaced cut-outs 183b′ defined through gripping portion 182′ of proximal handle portion 12a′ towards the proximal end of proximal handle portion 12a′. In addition to reducing the weight and the amount of material of proximal handle portion 12a′, cut-outs 183b′ are positioned to facilitate the cleaning and sterilization of proximal handle portion 12a′, particularly in and around finger 184′ and locator bar 186′ (FIG. 38), which will be described below. Proximal handle portion 12a′ may also include a release aperture 185′ defined therethrough at the proximal end thereof, the importance of which will be described below.

Referring again to FIGS. 2-3, cartridge receiving half-section 14 includes a central portion 173 which defines spaced centrally disposed U-shaped recesses 174 positioned to support lateral support members 172 of anvil half-section 12. A distal wall 173a of central portion 173 defines a tissue stop (FIG. 3). A pair of lateral cylindrical pivot members 187 are positioned beneath recesses 174. Each pivot member defines a flat 187a (FIG. 2a). The proximal end of cartridge receiving half-section 14 also includes a pair of vertical support members 188. Each vertical support member 188 includes an elongated vertical slot 188a having a rounded bottom surface. Vertical slots 188a are dimensioned to receive protrusions 186 formed on finger 184 of anvil half-section 12 (FIG. 21) when the anvil half-section 12 is supported on the cartridge receiving half-section 14 during assembly. By positioning protrusions 186 within vertical slots 188a, anvil half-section 12 can be pivoted in a scissor-like action with respect to the cartridge receiving half-section 14 between open and closed positions. In an embodiment, protrusions 186 have a tear drop profile. At least one sidewall of cartridge receiving half-section 14 includes a depression 189 (see FIG. 3) which will be discussed in further detail below.

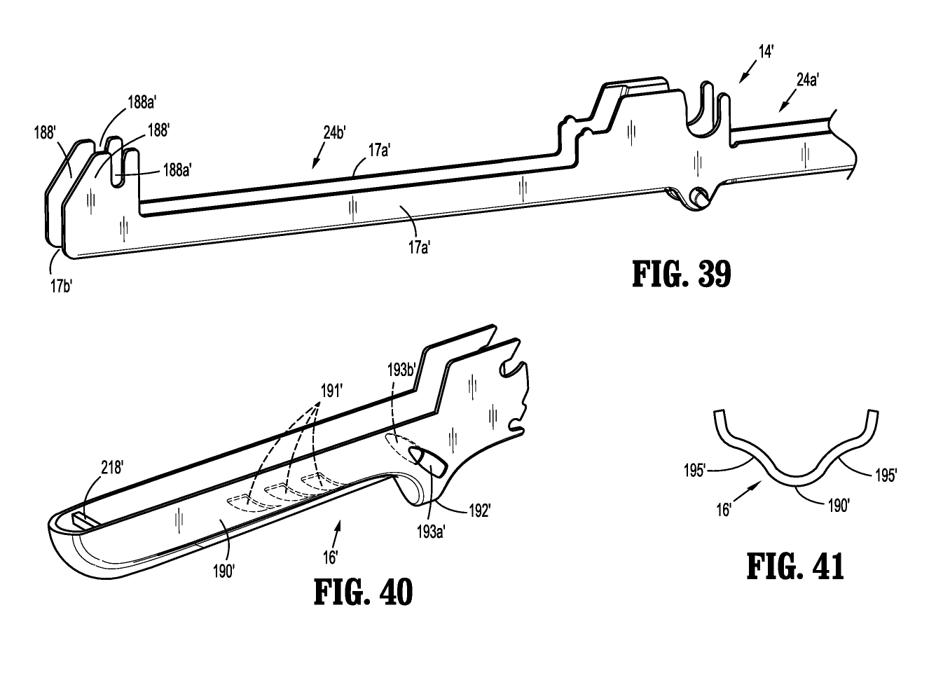

Referring to FIGS. 38-39, in an embodiment, as an alternative to finger 184 and protrusions 186 (FIGS. 2-3), a proximal end of proximal handle portion 12a′ includes a downwardly extending finger 184′ having a transversely-extending locator bar 186′ disposed at a free end of finger 184′ (FIG. 38). Finger 184′ and locator bar 186′ may be monolithically, i.e., integrally, formed with proximal handle portion 12a′. Locator bar 186′ is configured for receipt within vertical slots 188a′ of vertical support members 188′ of cartridge receiving half-section 14′ (FIG. 39).

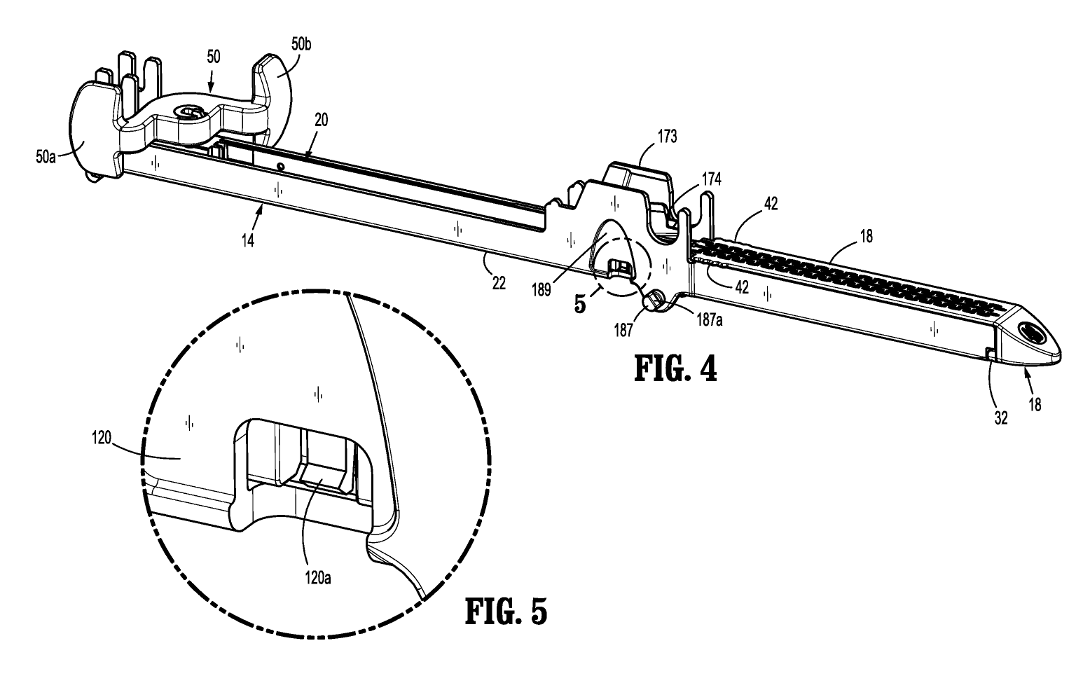

With continued reference to FIGS. 38-39, similar to cartridge receiving half-section 14 (FIGS. 3-7), cartridge receiving half-section 14′ defines a U-shaped configuration having a distal portion 24a′ dimensioned to releasably receive a SULU 18 (FIG. 4) and a proximal portion 24b′ dimensioned to releasably receive firing assembly 20′ (FIG. 42). Proximal portion 24b′ includes a bottom wall (not shown) and a pair of sidewalls 17a′. Sidewalls 17a′ extend proximally beyond the bottom wall (not shown) such that a proximal opening 17b′ is defined between sidewalls 17a′ adjacent the proximal ends of sidewalls 17a′, the importance of which will be described below. The proximal end of cartridge receiving half-section 14′ also includes a pair of vertical support members 188′, each including an elongated vertical slot 188a′ having a rounded bottom surface. Vertical slots 188a′, as mentioned above, are dimensioned to receive locator bar 186′ of proximal handle portion 12a′ of anvil half-section 12′ (FIG. 36) when the anvil half-section 12′ is supported on the cartridge receiving half-section 14′ during assembly.

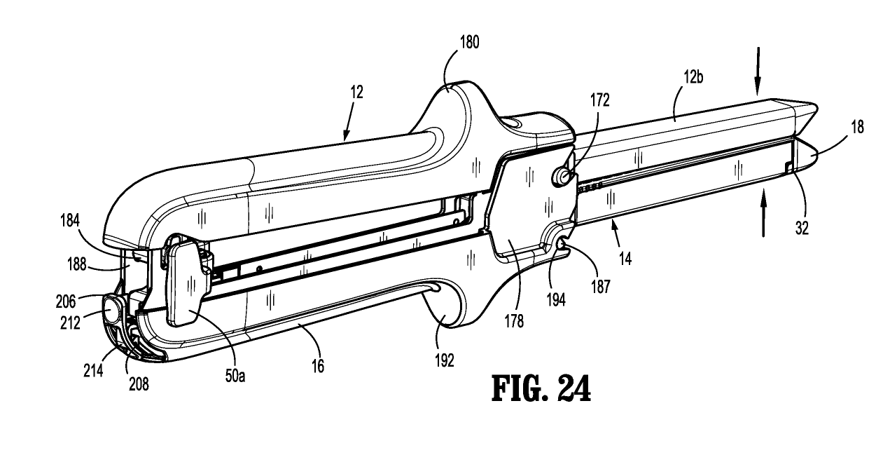

Referring back to FIGS. 2-3, clamping lever 16 also includes a handle portion 190 including a grip 190a and a finger engaging member, such as thumb engaging abutment 192. As discussed above, a pair of spaced flange portions 178 are supported on the distal end of clamping lever 16. Each flange portion 178 defines a cutout 176 dimensioned to receive a respective lateral support member 172 of anvil half-section 12 when stapler 10 is moved towards clamped position (FIG. 2B). The distal end of clamping lever 16 also defines a pair of distal C-shaped recesses 194 which are dimensioned to receive pivot members 187. Each recess 194 defines a mouth which is substantially smaller in width than the diameter of the pivot members 187. Because the mouth of each C-shaped recess 194 is smaller in width than the diameter of the pivot member 187, when clamp lever 16 is secured to cartridge receiving half-section 14 (FIG. 24), the pivot members 187 are slid into recesses 194 along the surface of flats 187a of the pivot members 187. As such, clamping lever 16 may be positioned as shown in FIG. 2A to pivotally secure clamping lever 16 about pivot members 187 of cartridge receiving half-section 14. After positioning pivot members 187 in C-shaped recesses 194, clamping lever 16 can be rotated in a counter-clockwise direction as shown in FIG. 2A to the position shown in FIG. 2 to secure clamp lever 16 to cartridge receiving half-section 14.

In embodiments, as shown in FIGS. 40 and 41, clamping lever 16′ may include a pair of opposed cut-outs 193a′, 193b′ defined through clamping lever 16′ adjacent thumb engaging abutment 192′. Clamping lever 16′ may additionally or alternatively include a plurality of longitudinally spaced-apart cut-outs 191′ defined through handle portion 190′ of clamping lever 16′ along at least a portion of the length of handle portion 190′. Cut-outs 193a′, 193b′ and 191′ facilitate cleaning and sterilization of clamping handle 16′, reduce the overall amount of material required to form clamping handle 16′, and reduce the weight of clamping handle 16′, while maintaining the requisite strength and functionality thereof.

Handle portion 190′ of clamping lever 16′ may further define a pair of elongated recesses 195′ extending longitudinally along either side of handle portion 190′ that are configured to facilitate the grasping of handle portion 190′ from either side of clamping lever 16′ during movement of clamping lever 16′ to the clamped position. Recesses 195′ also contribute to the reduced profile of clamping lever 16′.

As shown in FIG. 3A, an inner wall of clamping lever 16 includes a protrusion 201. As clamping lever 16 is rotated from the position shown in FIG. 2A to the position shown in FIG. 2B, protrusion 201 moves along and is deformed by an outer wall of cartridge receiving half-section 14 until protrusion 201 moves into depression 189 (FIG. 2A) formed in the sidewall of cartridge receiving half-section 14. By positioning protrusion 201 in depression 189, clamping lever 16 is prevented from rotating to a position in which the clamping lever 16 can be disengaged with the cartridge receiving half-section 14 (FIG. 2A) and is, thus, retained in the open position (FIG. 2B).

Referring to FIG. 2, in preparation for use, after clamping lever 16 has been secured to cartridge receiving half-section 14 and SULU 18 and firing assembly 20 are loaded into channel member 22, anvil half-section 12 can be assembled to cartridge receiving half-section 14. It is noted that SULU 18 and firing assembly 20 can be loaded into channel member 22 prior to or after securement of clamping lever 16 to cartridge receiving half-section 14. To attach anvil half-section 12 to cartridge receiving half-section 14, protrusions 186 of finger 184 are positioned in vertical slots 188a of vertical support member 188 of cartridge receiving half-section 14. Thereafter, anvil half-section 12 is rotated towards cartridge receiving half-section 14 to position lateral supports members 172 in U-shaped recesses 174. Once assembled in this manner, surgical stapler 10 may be positioned within a surgical site such that tissue is disposed between cartridge receiving half-section 14 and anvil half-section 12 for stapling and dividing the tissue.

In order to position surgical stapler 10 in the clamped position for clamping tissue between cartridge receiving half-section 14 and anvil half-section 12, clamping lever 16 is rotated in a counter-clockwise direction from the position shown in FIG. 2B. As clamping lever 16 is rotated, lateral support members 172 are received in cutouts 176 of flange portions 178 and cammed towards cartridge receiving half-section 14. As shown in FIG. 3, a spring member 200 is secured to an inner surface of clamping lever 16, such as by welding, at a position to engage cartridge receiving portion 14 to urge clamping lever 16 to the non-clamped position shown in FIG. 2B. In the clamped position shown in FIG. 1, the staple deforming portion 198 is positioned in close approximation with the top surface of SULU 18 with tissue held therebetween.

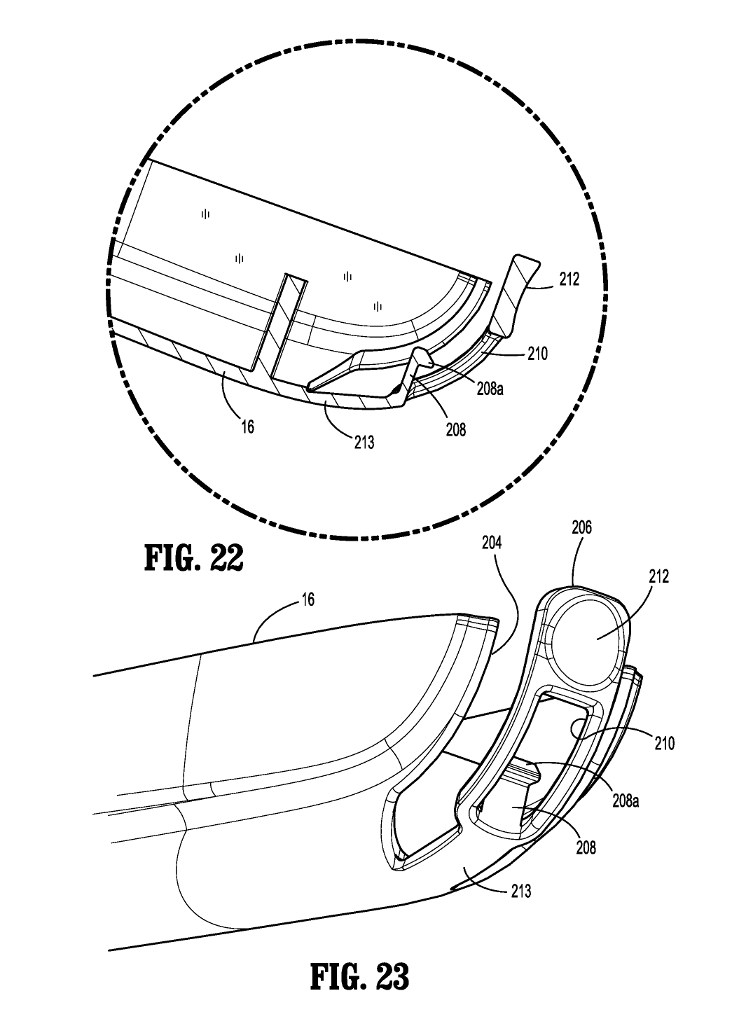

Referring to FIGS. 18 and 22-27, the proximal end of clamping lever 16 includes a cutout 204, a resilient cantilevered locking member 206 positioned within the cutout 204, and a resilient engagement member 208 supported on the locking member 206. Locking member 206 defines an opening 210 and a finger engagement surface 212. Engagement member 208 defines a hook portion which is positioned within opening 210 and includes a locking surface 208a. In an embodiment, the cantilevered locking member 206 is integrally formed with clamping lever 16 and is connected to clamping lever 16 by a living hinge 213 (FIG. 23). Referring to FIGS. 3, 25 and 27, the proximal end of cartridge receiving half-section 14 includes a catch member 214 which extends downwardly from cartridge receiving half-section 14. In an embodiment, catch member 214 is integrally formed with half-section 14 and has a transverse locking surface 214a. Catch member 214 is positioned to be received within opening 210 and to engage and deflect engagement member 208 inwardly when clamping lever 16 is moved to the clamped position such that locking surface 208a of engagement member 208 automatically snaps into releasable engagement with locking surface 214a when clamp lever 16 is moved to the clamped position to retain clamping lever 16 in the clamped position. To accomplish this, locking member 214 and engagement member 208 define angled cam surfaces which abut during movement of the clamping lever 16 to the clamped position to deflect engagement member 208 inwardly past catch member 214. In order to release engagement member 208 from catch member 214, cantilevered locking member 206 can be depressed by pressing on finger engagement surface 212 to move engagement member 208 inwardly out of engagement with catch member 214. When this occurs, spring member 200 automatically urges clamping lever 16 to the unclamped position.

Referring to FIGS. 3, 12, 19 and 26, as discussed above, guide block 48 is pivotally supported in stationary housing 26 of firing assembly 20. Guide block 48 includes a distally extending nose portion 220 (FIGS. 12 and 26) which rests beneath SULU 18 when SULU 18 is supported in channel member 22. The internal surface of guide block 48 includes locking surfaces 222 (FIG. 19) which are received in notches 49 and 51 of knife actuating bar 44 when the stapler 10 is in an unclamped position. When the SULU 18 is positioned in the channel member 22, prior to moving clamp lever 16 to the clamped position, SULU 18 is positioned atop nose portion 220 and is not fully seated in the channel member 22, as discussed above. When the stapler 10 is moved to the clamped position, locating fingers 170 of anvil half-section 12 (FIG. 3) engage a top surface of body 120 of SULU 18 to fully seat SULU 18 in channel member 22. Locating fingers 170 engage SULU 18 to properly position SULU 18 in relation to anvil portion 12b. As SULU 18 is fully seated in channel member 22, SULU 18 presses downwardly on nose portion 220 of guide block 48 to pivot guide block 48 about protrusions 72. When guide block 48 pivots, locking surfaces 222 move from notches 49 and 51 to unlock knife actuating bar 44 (FIG. 26). This configuration prevents movement of the knife actuating bar 44 in relation to guide block 48 prior to clamping to ensure that the knife actuating bar 44 and SULU knife 40 remain properly positioned for operational engagement prior to use.

With reference to FIGS. 35, 36, 39-40, and 42, as an alternative to the locking structure of surgical stapler 10 (FIGS. 1-34), a pivotal locking member 206′ (FIG. 42) may be pivotally supported on the proximal end of stationary housing 26′ of firing assembly 20′. Pivotal locking member 206′ includes pivot members 29′ which are pivotably disposed within openings 28′ defined in the sidewalls 30a′ of stationary housing 26′. A release member 212′ is positioned on one end of locking member 206′ and a latch portion 210′ is positioned on the other end of locking member 206′. Latch portion 206′ includes a hook member 210a′. A biasing member 214′ is positioned about one or both of pivot members 29′ to urge latch portion 210′ inwardly towards stationary housing 26′ of firing assembly 20′. When locking member 206′ is secured on firing assembly 26′, latch portion 210′ extends downwardly through an opening 31′ (FIG. 42) in a proximal end of bottom wall 30b′ of stationary housing 26′ and through proximal opening 17b′ (FIG. 39) defined between sidewalls 17a′ of proximal portion 24b′ of cartridge receiving half-section 14′ to a position below the bottom wall (not shown) of proximal portion 24b′ of cartridge receiving half-section 14′.

A proximal end of clamping lever 16′ (FIG. 40) includes an engagement member 218′, which is positioned to be engaged by hook member 210a′ of latch portion 210′ when the clamp lever 16′ is moved to the clamped position. More specifically, as clamping lever 16′ is moved towards the clamped position, locking member 206′ is pivoted about pivot members 29′ such that hook member 210a′ of latch portion 210′ passes and snaps into engagement with engagement member 218′.

Proximal handle portion 12a′ of anvil half-section 12′, as mentioned above, may include a release aperture 185′ (FIG. 36) that is configured to at least partially receive release member 212′ when clamping lever 16′ is disposed in the clamped position such that release member 212′ is accessible from the exterior of proximal handle portion 12a′. Thus, to release latch portion 210′ from engagement member 218′, the operator inserts a finger into release aperture 185′ to depress release member 212′ of locking member 206′, thereby pivoting latch portion 210′ out of engagement with engagement member 218′. When this occurs, spring member 200′ (FIG. 42) urges clamping lever 16′ to the unclamped position.

Referring to FIGS. 24-28, when stapler 10 is in the clamped, unfired position grasping tissue between cartridge receiving half-section 14 and anvil half-section 12, slide block 52 of firing assembly 20 is in the retracted position at the proximal end of channel member 22 and stationary housing 26. See FIG. 27. In this position, pedal 54 is positioned in cutout 55 of channel member 22 and pin 88 of pedal 54 is positioned in arcuate recess 94 of firing lever 50 beneath stop recesses 94a. As such, firing lever 50 can be pivoted to facilitate actuation of stapler 10 from either side of the stapler 10. In addition, in this position of slide block 52, finger 108 of knife actuating bar 44 is positioned adjacent the distal wall of recess 110 of slide block 52. Engagement member 208 is also engaged with locking member 214 to retain clamping lever 16 in the clamped position.

Referring to FIG. 26, when slide block 52 is in the retracted position, knife 40 and cam surfaces 114b of cam bar 46 are positioned in the proximal end of SULU 18 and, proximal hook 156 of safety lockout 128 is positioned in engagement with engagement member 158 of knife 40 to retain safety lockout 128 in the locked orientation. In addition, downturned hook portion 40b of knife 40 is engaged with upturned hook portion 106 of knife actuating bar 44 to connect firing assembly 20 to knife 40 of SULU 18.

Referring to FIGS. 29-32, when the firing lever 50 is advanced distally in the direction indicated by arrow “A” in FIG. 29, slide block 52 is moved distally within stationary housing 26 of firing assembly 20 to effect corresponding movement of cam bar 46 and delayed movement of knife actuating bar 44. As discussed above, the delayed movement of the knife actuating bar 44 is equal to the length of recess 110 of slide block 52 and results from movement of finger 108 of knife actuating bar 44 within recess 110 of slide block 52. Movement of knife actuating bar 44 with slide block 52 begins when finger 108 abuts the proximal wall 112 of recess 110. As cam bar 46 is moved distally through stationary housing 26 of firing assembly 20, cam surfaces 114b on sidewalls 114 of cam bar 46 are advanced through SULU 18 to sequentially engage pushers 122 to eject staples 126 from slots 130 of body 120, through the tissue, and into the staple deforming portion 198 for forming staples 126 about tissue in a plurality of staple lines. Concurrently, since the distal end of knife actuating bar 44 is engaged with knife 40, knife 40, after the preset delay, is advanced through SULU 18 to incise the tissue between the staple lines.

As shown in phantom in FIG. 32, when slide block 52 moves distally within stationary housing 26, pedal 54 rides up over channel member 22 and moves along inner surface of stationary housing 26 of firing assembly 20. When this occurs, pin 88 of pedal 54 moves into a stop recess 94a to prevent further pivotal movement of firing lever 50.

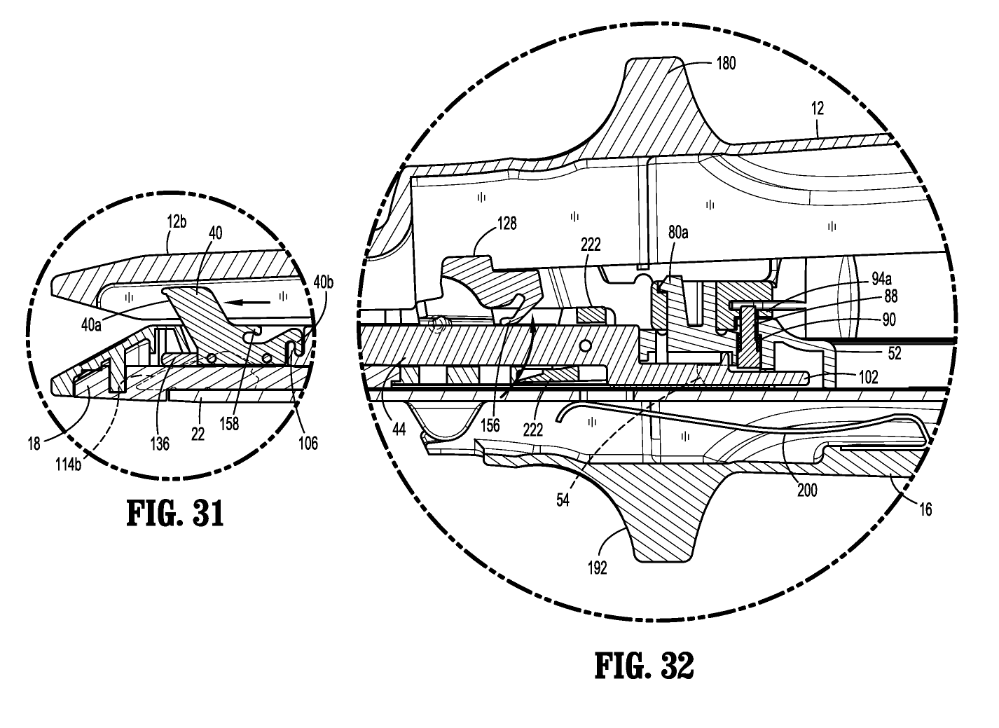

Referring to FIGS. 31 and 32, when knife 40 is moved distally within SULU 18, engagement member 158 of knife 40 is disengaged with proximal hook 156 of safety lockout 128.

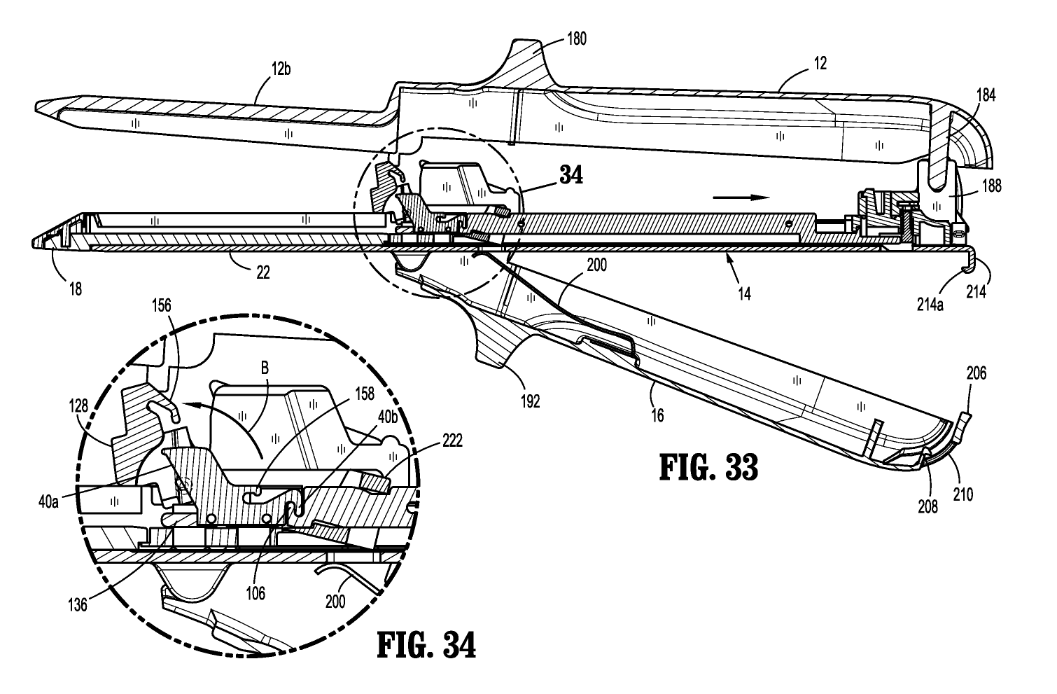

Referring to FIGS. 33 and 34, when the firing lever 50 is returned to its proximal-most position to retract cam bar 46 and knife 40, and the cantilevered locking member 206 is depressed to disengage engagement member 208 from catch member 214, spring 200 urges clamping lever 16 to its unclamped position to allow stapler 10 to move to the open position, releasing the stapled and divided tissue. In the open position, anvil half-section 12 is spaced from cartridge receiving half-section 14 and spring 152 (FIG. 17) pivots safety lockout 128 in the direction indicated by arrow B in FIG. 34 about pivot member 150 to its unlocked position such that safety lockout 128 projects upwardly from SULU 18. In the unlocked position, safety lockout 128 prevents movement of the stapler 10 back to the clamped position. In order to reuse stapler 10, used SULU 18 must be replaced with a new SULU 18.

During a surgical procedure, SULU 18 can be replaced multiple times to facilitate multiple uses of stapler 10 on a single patient. For example, during a surgical procedure to remove a diseased portion of a vessel from the vessel, the surgical stapler 10 can be used to staple and cut tissue on opposite sides of the diseased vessel portion. The SULU 400 can be replaced after each staple and cut operation of the surgical stapler 10. Since each SULU 18 is provided with a fresh knife 40, tearing of tissue is minimized. After the surgical procedure, the used SULU(S) 18 and the firing assembly 20 can be removed from the channel member 22 and disposed of in an appropriate manner. Thereafter, clamping lever 16 can be removed from cartridge receiving half-section 14, by rotating clamping lever 16 to the position shown in FIG. 2A and disengaging pivot members 187 from C-shaped recesses 194. The anvil half-section 12, cartridge receiving half-section 14 and clamping lever 16 can now be sterilized, such as by autoclaving, and reused with a sterilized SULU 18 and firing assembly 20 in the manner discussed above.

It will be understood that various modifications may be made to the embodiments of the surgical fastener applying apparatus disclosed herein. Therefore, the above description should not be construed as limiting, but merely as exemplifications of embodiments. Those skilled in the art will envision other modifications within the scope and spirit of the present disclosure.

What is claimed is:

1. A surgical fastener applying apparatus comprising:
an anvil half-section including a distal anvil portion and a proximal handle portion, the proximal handle portion defining a release aperture at the proximal end of the proximal handle portion;
a cartridge receiving half-section having a distal portion dimensioned to receive a single use loading unit and a proximal portion configured to support a firing assembly;
a clamping lever secured to the cartridge receiving half-section, the clamping lever having a proximal end and a distal end and including a handle portion supporting an engagement member; and
a firing assembly configured to be supported within the proximal portion of the cartridge receiving half-section and including a stationary housing, a firing lever, a cam bar fixedly secured to the firing lever, and a pivotal locking member including a latch portion and a release member;
the clamping lever being operably associated with the anvil half-section and the cartridge receiving half-section and being movable from an unclamped position to a clamped position to releasably secure the anvil portion of the anvil half-section in close approximation with the single use loading unit, wherein in the clamped position, the engagement member of the clamping lever releasably engages the latch portion of the locking member and the release member extends at least partially into the release aperture.

2. The surgical fastener applying apparatus according to claim 1, wherein the pivotal locking member includes a pair of pivot members which are pivotally supported in openings defined in a proximal end of the stationary housing of the firing assembly.

3. The surgical fastener applying apparatus according to claim 2, wherein the release member and the latch portion of the pivotal locking member are disposed on opposite ends of the pivotal locking member and wherein the pivot members are disposed between the release member and the latch portion.

4. The surgical fastener applying apparatus according to claim 1, wherein the proximal handle portion of the anvil half-section defines at least one cut-out adjacent the release aperture.

5. A surgical fastener applying apparatus comprising:
an anvil half-section including a distal anvil portion and a proximal handle portion, the distal anvil portion defining a longitudinally-extending knife slot and a transversely-extending slot, the knife slot and the transverse slot disposed in substantially perpendicular orientation relative to one another and communicating with one another;
a cartridge receiving half-section having a distal portion dimensioned to receive a single use loading unit and a proximal portion configured to support a firing assembly;
a clamping lever secured to the cartridge receiving half-section; and
a firing assembly configured to be supported within the proximal portion of the cartridge receiving half-section and including a stationary housing, a firing lever, a cam bar fixedly secured to the firing lever, and a knife actuating bar which is configured to engage a knife supported within the single use loading unit;
the clamping lever being operably associated with the anvil half-section and the cartridge receiving half-section and being movable from an unclamped position to a clamped position to releasably secure the anvil portion of the anvil half-section in close approximation with the single use loading unit, wherein in the clamped position, the firing lever is selectively advancable to advance the cam bar to eject staples from the single use loading unit and the knife actuating bar to translate the knife through the knife slot.

6. The surgical fastener applying apparatus according to claim 5, wherein the proximal handle portion of the anvil half-section defines a plurality of longitudinally spaced cut-outs disposed along at least a portion of a length thereof.

7. The surgical fastener applying apparatus according to claim 5, wherein the clamping lever includes a thumb abutment disposed towards the distal end thereof, the clamping lever further including a pair of opposed cut-outs defined therethrough adjacent the thumb abutment.

8. A surgical fastener applying apparatus comprising:
an anvil half-section including a distal anvil portion and a proximal handle portion;
a cartridge receiving half-section having a distal portion dimensioned to receive a single use loading unit and a proximal portion configured to support a firing assembly;
a clamping lever secured to the cartridge receiving half-section, the clamping lever being operably associated with the anvil half-section and the cartridge receiving half-section and being movable from an unclamped position to a clamped position to releasably secure the anvil portion of the anvil half-section in close approximation with the single use loading unit; and
a firing assembly configured to be supported within the proximal portion of the cartridge receiving half-section and including a stationary housing, a firing lever, and a cam bar fixedly secured to the firing lever, the firing lever being distally translatable from a proximal position to a distal position to advance the cam bar to eject staples from the single use loading unit and proximally translatable from the distal position to the proximal position to return the cam bar back to an initial position, the firing lever including an engagement member having a first concave surface configured to facilitate finger engagement of the firing lever during distal translation of the firing lever and a second, opposed concave surface configured to facilitate finger engagement of the firing lever during proximal translation of the firing lever.

9. The surgical fastener applying apparatus according to claim 8, wherein the firing assembly further includes a knife actuating bar which is configured to engage a knife supported within the single use loading unit to translate the knife through the single use loading unit upon translation of the firing lever.

10. The surgical fastener applying apparatus according to claim 8, wherein the firing lever includes a pair of engagement members, a first engagement member extending from a first side of the firing assembly and a second engagement member extending from a second, opposite side of the firing assembly.
